(19) United States
(12) Patent Application Publication
(10) Pub. No.: US 2023/0014132 A1
(43) Pub. Date: Jan. 19, 2023

(54) LOOP-MEDIATED ISOTHERMAL AMPLIFICATION (LAMP) ANALYSIS FOR PATHOGENIC TARGETS

(71) Applicants: Purdue Research Foundation, West Lafayette, IN (US); Raytheon BBN Technologies, Corp., Cambridge, MA (US)

(72) Inventors: Jordan Seville, Delran, NJ (US); Darby McChesney, Lawrenceville, NJ (US); Josiah Davidson, West Layfayette, IN (US); Jiangshan Wang, West Lafayette, IN (US); Murali Kannan Maruthamuthu, Lafayette, IN (US); Andres Dextre, West Lafayette, IN (US); Mohit Verma, West Lafayette, IN (US)

(21) Appl. No.: 17/576,971

(22) Filed: Jan. 16, 2022

Related U.S. Application Data
(60) Provisional application No. 63/138,310, filed on Jan. 15, 2021.
(60) Provisional application No. 63/138,312, filed on Jan. 15, 2021.
(60) Provisional application No. 63/138,314, filed on Jan. 15, 2021.
(60) Provisional application No. 63/139,316, filed on Jan. 20, 2021.
(60) Provisional application No. 63/138,318, filed on Jan. 15, 2021.
(60) Provisional application No. 63/138,320, filed on Jan. 15, 2021.
(60) Provisional application No. 63/138,321, filed on Jan. 15, 2021.
(60) Provisional application No. 63/138,323, filed on Jan. 15, 2021.
(60) Provisional application No. 63/138,337, filed on Jan. 15, 2021.
(60) Provisional application No. 63/138,341, filed on Jan. 15, 2021.
(60) Provisional application No. 63/148,527, filed on Feb. 11, 2021.

(51) Int. Cl. C12Q 1/6806; C12Q 1/6844; C07K 14/08

(52) U.S. Cl. CPC C12Q 1/6806; C12Q 1/6844; C07K 14/08

(57) ABSTRACT

The present disclosure is drawn to compositions, methods, and systems for loop-mediated isothermal amplification (LAMP) analysis on a solid phase medium. The composition can comprise one or more target primers, a DNA polymerase, and a re-solubilization agent. The composition can be substantially free of non-pH sensitive agents capable of discoloring the solid phase medium. The method can comprise providing an assembly of a solid phase medium, depositing a biological sample onto the solid phase medium, and heating the assembly to an isothermal temperature sufficient to facilitate a LAMP reaction. The system can comprise a composition and a solid phase medium on to which the composition is deposited.

### RELATED APPLICATIONS

This application claims the benefit of U.S. Provisional Patent Application Ser. No. 63/138,310 filed Jan. 15, 2021, U.S. Provisional Patent Application Ser. No. 63/138,312 filed Jan. 15, 2021, U.S. Provisional Patent Application Ser. No. 63/138,314 filed Jan. 15, 2021, U.S. Provisional Patent Application Ser. No. 63/138,316 filed Jan. 15, 2021, U.S. Provisional Patent Application Ser. No. 63/138,318 filed Jan. 15, 2021, U.S. Provisional Patent Application Ser. No. 63/138,320 filed Jan. 15, 2021, U.S. Provisional Patent Application Ser. No. 63/138,321 filed Jan. 15, 2021, U.S. Provisional Patent Application Ser. No. 63/138,323 filed Jan. 15, 2021, U.S. Provisional Patent Application Ser. No. 63/138,337 filed Jan. 15, 2021, U.S. Provisional Patent Application Ser. No. 63/138,341 filed Jan. 15, 2021, U.S. Provisional Patent Application Ser. No. 63/148,527 filed Feb. 11, 2021, the entire contents of each of which are incorporated herein by reference.

### BACKGROUND

Polymerase chain reaction (PCR) is a molecular biology technique that allows amplification of nucleotides for various analytical purposes. Quantitative PCR (qPCR) is an adaptation of PCR which allows monitoring of the amplification of a targeted nucleotide. Diagnostic qPCR has been applied to detect nucleotides that are indicative of infectious diseases, cancer, and genetic abnormalities. Reverse transcription PCR (RT-PCR) is an adaptation of qPCR which allows detection of a target RNA nucleotides. Because of this ability, RT-PCR is well-suited for detecting virus pathogens. However, RT-PCR uses sizeable equipment which may not be available in certain point of care settings. Additionally, RT-PCR uses trained personnel, significant sample preparation, and time to perform and obtain results.

By contrast, Loop-Mediated Isothermal Amplification (LAMP) is a more simplistic approach to diagnostic identification of target nucleotides. In particular, LAMP is a one-operation nucleic acid amplification method to multiply specific nucleotide sequences. In addition to use of an isothermal heating process, LAMP can use a simple visual output test indicator, such as a color change rather than a more complicated fluorescent indicator used by PCR. Reverse-transcription LAMP (RT-LAMP) can be used like RT-PCR in order to identify target nucleotides from RNA, and as such, can be used in a diagnostic capacity to identify the presence or absence of viral pathogens. Because LAMP is more simplistic, it can be performed with less equipment and sample preparation and therefore is more accessible for use in point of care settings, such as clinics, emergency rooms, and even on a mobile basis.

### SUMMARY

The present disclosure is drawn to technology (e.g., compositions, methods, systems, and assemblies) for use in detecting a target nucleotide using a LAMP analysis. In some aspects, the target nucleotide can be known to reside in a pathogen of interest. In cases where the pathogen is a virus, the LAMP analysis can be an RT-LAMP analysis.

In some disclosure embodiments methods of preparing saliva samples for loop-mediated isothermal amplification (LAMP) detection of a pathogen target are provided. In one aspect, such a method can include providing an amount of saliva from a test subject, and diluting the saliva in water to a degree that reduces a buffering capacity of the saliva while maintaining a sufficient concentration to allow for detection of the pathogen target.

In one aspect, the method can include reducing a viscosity of the saliva as compared to an original viscosity. In another aspect, the viscosity can be reduced by one or more of dilution, filtering, or combinations thereof. In another aspect, the viscosity can be reduced using filtering. In a further aspect, the viscosity can be reduced using a 10-micron filter. In yet another aspect, the viscosity can be reduced to a degree that increases flowability through a solid phase medium as compared to an original viscosity. In yet another aspect, the viscosity can be reduced to a range of from about 1.0 centipoise (cP) to about 50 cP.

In one aspect, such a method can include filtering the saliva sample to a degree that adjusts a saliva sample pH to a test sample target range. In another aspect, the test sample target range can be from about 7.2 to about 8.6. In another aspect, the water can have a pH greater than 6.0 and can be substantially free of contaminants. In yet another aspect, the saliva sample can consist essentially of saliva and water. In yet a further aspect, the saliva can be collected using sponge-based collection.

In one aspect, the saliva can be diluted in the water to a saliva to water ratio of about 1:1 to about 1:20. In another aspect, the saliva can be diluted in the water to a degree that provides the sample with an optical density at 600 nm (OD₆₀₀) of less than 0.2. In a further aspect, the saliva has a volume from about 50 μl to about 100 μl. In yet another aspect, the saliva sample has a volume ranging from about 100 μl to about 1 ml.

In an additional aspect, the pathogen target can comprise a viral pathogen, a bacterial pathogen, a fungal pathogen, or a protozoa pathogen. In one aspect, the pathogen target can be a viral target. In another aspect, the viral target can comprise a dsDNA virus, an ssDNA virus, a dsRNA virus, a positive-strand ssRNA virus, a negative-strand ssRNA virus, an ssRNA-RT virus, or a ds-DNA-RT virus. In yet another aspect, the viral target can be H1N1, H2N2, H3N2, H1N1pdm09, or SARS-CoV-2.

In some aspects, the LAMP detection can comprise reverse transcription LAMP (RT-LAMP) detection.

In other disclosure embodiments, test sample compositions for LAMP analysis are disclosed and can include: an amount of a test subject's saliva that is sufficient to detect a pathogen target via a LAMP analysis in combination with an amount of water that reduces a buffering capacity of the saliva.

In one aspect, the composition can have a viscosity of from about 1.0 cP to about 50 cP. In another aspect, the composition can have a pH of from about 7.2 to about 8.6. In another aspect, the composition can have a saliva to water ratio of about 1:1 to about 1:20. In yet another aspect, the composition can have an optical density at 600 nm (OD₆₀₀) of less than 0.2. In another aspect, the water can have a pH greater than 6.0 and can be substantially free of contaminants. In one aspect, the composition can consist essentially of saliva and water. In another aspect, the saliva can have a volume ranging from about 50 μl to about 100 μl. In yet another aspect, the saliva sample can have a volume of from about 100 μl to about 1 ml.

In one aspect, the pathogen target can comprise a viral pathogen, a bacterial pathogen, a fungal pathogen, or a protozoa pathogen. In another aspect, the pathogen target can be a viral target. In another aspect, the viral target can comprise a dsDNA virus, an ssDNA virus, a dsRNA virus, a positive-strand ssRNA virus, a negative-strand ssRNA virus, an ssRNA-RT virus, or a ds-DNA-RT virus. In yet another aspect, the viral target can comprise H1N1, H2N2, H3N2, H1N1pdm09, or SARS-CoV-2. In yet another aspect, the buffering capacity of the composition can be less than 5 mM.

In yet other disclosure embodiments, compositions for LAMP analysis on a solid phase medium can include one or more target primers, a DNA polymerase, and a re-solubilization agent. In some aspects, such a composition can be substantially free of non-pH sensitive agents capable of discoloring the solid phase medium. In one aspect, the composition can include an antioxidant. In another aspect, the composition can be substantially free of volatile agents. In yet another aspect, the composition can be substantially free of hygroscopic agents. In one other aspect, the composition can further include reverse transcriptase.

In one aspect, the hygroscopic agents can absorb more than about 10 wt % when between about 40% and about 90% relative humidity (RH) at 25° C. In another aspect, the hygroscopic agents can include glycerol, ethanol, methanol, calcium chloride, potassium chloride, calcium sulfate, and combinations thereof.

In another aspect, the re-solubilization agent can be a surfactant. In another aspect, the re-solubilization agent can comprise bovine serum albumin (BSA), casein, polysorbate 20, or combinations thereof.

In one aspect, the target primers can target a pathogen that can comprise a viral pathogen, a bacterial pathogen, a fungal pathogen, or a protozoa pathogen. In one aspect, the pathogen can be a viral pathogen. In another aspect, the viral pathogen can comprise a dsDNA virus, an ssDNA virus, a dsRNA virus, a positive-strand ssRNA virus, a negative-strand ssRNA virus, an ssRNA-RT virus, or a ds-DNA-RT virus. In another aspect, the viral pathogen can comprise H1N1, H2N2, H3N2, H1N1pdm09, or SARS-CoV-2.

In one aspect, the composition can further comprise a non-discoloration additive. The non-discoloration additive can comprise one or more of a sugar, a buffer, or combinations thereof. In another aspect, the composition can further comprise an indicator.

In other disclosure embodiments, a method for LAMP analysis on a solid phase medium can include providing an assembly of a solid phase medium and a composition as recited herein, depositing a biological sample onto the solid phase medium, and heating the assembly to an isothermal temperature sufficient to facilitate a LAMP reaction. In one aspect, the biological sample can be one or more of saliva, mucus, blood, urine, feces, sweat, exhaled breath condensate, or combinations thereof. In another aspect, the biological sample is saliva. In one aspect, the LAMP analysis can be reverse transcriptase LAMP (RT-LAMP). In another aspect, the method can further comprise detecting a viral pathogen.

In other disclosure embodiments, a system for performing the LAMP analysis can comprise a composition as recited herein, and a solid phase medium on to which the composition is deposited.

In yet further disclosure embodiments, compositions for loop-mediated isothermal amplification (LAMP) analysis can utilize a pH-dependent output signal that can include a pH sensitive dye, and a plurality of non-interfering LAMP reagents. In one aspect, the LAMP analysis can be RT-LAMP.

In one aspect, the pH sensitive dye can be at least one of phenol red, phenolphthalein, azolitmin, bromothymol blue, naphtholphthalein, cresol red, or combinations thereof. In another aspect, the plurality of non-interfering LAMP reagents can be substantially free of volatile reagents, pH-interfering reagents, magnesium-interfering reagents, or combinations thereof.

In one aspect, the plurality of non-interfering LAMP reagents can be substantially free of magnesium, ammonium sulfate, and ammonium carbonate. In aspect, the plurality of non-interfering LAMP reagents can comprise DNA polymerase, reverse transcriptase, target primers, or combinations thereof.

In another aspect, the composition can comprise an antioxidant. In another aspect, the composition can further comprise carrier RNA, carrier DNA, RNase inhibitors, DNase inhibitors, guanidine hydrochloride, or combinations thereof. In one aspect, the composition can further comprise a solid phase medium.

In one aspect, the composition can comprise a non-discoloration additive that can comprise a sugar, a buffer, a blocking agent, or combinations thereof. In one aspect, the sugar can comprise one or more of trehalose, glucose, sucrose, or combinations thereof. In another aspect, the blocking agent can comprise bovine serum albumin, casein, or combinations thereof.

In other disclosure embodiments, methods of performing a LAMP analysis with a pH-dependent output signal are provided and can include providing an assembly of a solid phase medium and a composition as recited herein, depositing a biological sample onto the solid phase medium, and heating the assembly to an isothermal temperature sufficient to facilitate a LAMP reaction. In one aspect, the LAMP analysis can be RT-LAMP. In one aspect, the biological sample can be one or more of saliva, mucus, blood, urine, feces, sweat, exhaled breath condensate, and combinations thereof. In one aspect, the biological sample can be saliva. In another aspect, the method can further comprise detecting a viral pathogen.

In further disclosure embodiments, methods of maximizing accuracy of an output signal in a pH-dependent LAMP analysis can comprise providing a reagent mixture that minimizes non-LAMP reaction produced discoloration from a signal output medium, and performing the LAMP reaction. In one aspect, the method can comprise controlling production of protons from a non-LAMP reaction. In another aspect, the method can comprise controlling oxidation from a non-LAMP reaction.

In other disclosure embodiments, a method of maximizing accuracy of an output signal in a pH-dependent LAMP analysis can comprise substantially eliminating non-LAMP reaction produced discoloration from a signal output medium.

In other disclosure embodiments, methods of maximizing a limit of detection (LOD) in a pH-dependent LAMP analysis can include substantially eliminating non-LAMP reaction produced discoloration from a signal output medium.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure; and, wherein:

FIG. 1 depicts a method of preparing a saliva sample for loop-mediated isothermal amplification (LAMP) detection of a pathogen target in accordance with an example embodiment;

FIG. 2 depicts a method for LAMP analysis in accordance with an example embodiment;

FIG. 3 depicts a method of maximizing accuracy of an output signal in a pH-dependent LAMP analysis in accordance with an example embodiment;

FIG. 4 illustrates that loop-mediate isothermal amplification (LAMP) can be obtained in a saliva sample in accordance with an example embodiment;

FIG. 5A illustrates a sponge-based collection device in accordance with an example embodiment;

FIG. 5B illustrates a passive drool collection device in accordance with an example embodiment;

FIG. 6A illustrates the limit of detection for various concentrations of sample and various collection devices in accordance with an example embodiment;

FIG. 6B illustrates the effect of RNase inhibitors for various concentrations of template on the RT-LAMP colorimetric response in accordance with an example embodiment;

FIG. 6C illustrates the effect of the saliva processing technique on the colorimetric LoD in accordance with an example embodiment;

FIG. 6D illustrates the effect of carrier DNA concentration on colorimetric RT-LAMP response in accordance with an example embodiment;

FIG. 6E illustrates the effect of Guanidine HCl on RT-LAMP colorimetric response in accordance with an example embodiment;

FIG. 6F illustrates the effect of UDG on end-point RT-LAMP colorimetric response response in accordance with an example embodiment;

FIG. 6G illustrates the effect of saliva processing on colorimetric response in accordance with an example embodiment;

FIG. 7 is a chart illustrating the stability of frozen saliva samples in accordance with an example embodiment;

FIG. 8 illustrates the limit of detection of fresh saliva in accordance with an example embodiment;

FIG. 9 illustrates the limit of detection in a bovine nasal swab in accordance with an example embodiment;

FIG. 10 illustrates the limit of detection on paper in accordance with an example embodiment;

FIG. 11 illustrates the colorimetric transition for phenol red in accordance with an example embodiment;

FIG. 12 illustrates buffer used for the paper-based assay in accordance with an example embodiment;

FIG. 13A illustrates paper LAMP validation in accordance with an example embodiment;

FIG. 13B illustrates paper LAMP validation in accordance with an example embodiment;

FIG. 14A illustrates low template concentration LAMP on paper at a 0-minute time point in accordance with an example embodiment;

FIG. 14B illustrates low template concentration LAMP on paper at a 60-minute time point in accordance with an example embodiment;

FIG. 15 illustrates whole untreated saliva with heat inactivated SARS-CoV-2 virus in accordance with an example embodiment;

FIG. 16 illustrates a comparison of colorimetric and fluorometric RT-LAMP responses in accordance with an example embodiment;

FIG. 17A illustrates use of calmagite as a LAMP colorimetric indicator in accordance with an example embodiment;

FIG. 17B illustrates use of EBT as a LAMP indicator in accordance with an example embodiment;

FIG. 17C illustrates LAMP on chromatography paper using EBT as a colorimetric reporter in accordance with an example embodiment;

FIG. 17D illustrates colorimetric response of LAMP on various papers using EBT as an indicator in accordance with an example embodiment;

FIG. 17E illustrates LAMP detection on biodyne A amphoteric paper using EBT as a colorimetric indicator in accordance with an example embodiment;

FIG. 17F illustrates the effect of crystal violet concentration on LAMP colorimetric response in accordance with an example embodiment;

FIG. 17G illustrates colorimetric LAMP using crystal violet at various concentration on paper in accordance with an example embodiment;

FIG. 17H illustrates pH indicators as colorimetric reporters for RT-LAMP in accordance with an example embodiment;

FIG. 17I illustrates the effect of cresol red concentration on colorimetric response of LAMP reaction in accordance with an example embodiment;

FIG. 17J the effect of concentration of various pH indicators on colorimetric response for RT-LAMP reaction in accordance with an example embodiment;

FIG. 17K gel electrophoresis scans of RT-LAMP products using pH indicators in accordance with an example embodiment;

FIG. 17L the effect of initial pH on RT-LAMP colorimetric response using Phenol red in accordance with an example embodiment;

FIG. 18 illustrates the color stability of the drying process in accordance with an example embodiment;

FIG. 19A illustrates the effect of elimination of single reactant on initial color of paper after drying in accordance with an example embodiment; and

FIG. 19B illustrates the effect of trehalose and Tween 20 on RT-LAMP colorimetric response in accordance with an example embodiment.

Reference will now be made to the exemplary embodiments illustrated, and specific language will be used herein to describe the same. It will nevertheless be understood that no limitation of the scope of the technology is thereby intended.

### DESCRIPTION OF EMBODIMENTS

Before invention embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting. The same reference numerals in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of compositions, dosage forms, treatments, etc., to provide a thorough understanding of various invention embodiments. One skilled in the relevant art will recognize, however, that such detailed embodiments do not limit the overall inventive concepts articulated herein, but are merely representative thereof.

### Definitions

It should be noted that as used herein, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients.

As used herein, the term “soluble” is a measure or characteristic of a substance or agent with regards to its ability to dissolve in a given solvent. The solubility of a substance or agent in a particular component of the composition refers to the amount of the substance or agent dissolved to form a visibly clear solution at a specified temperature such as about 25° C. or about 37° C.

As used herein, the term “lipophilic,” refers to compounds that are not freely soluble in water. Conversely, the term “hydrophilic” refers to compounds that are soluble in water.

As used herein, a “subject” refers to an animal. In one aspect the animal may be a mammal. In another aspect, the mammal may be a human.

As used herein, “non-liquid” when used to refer to the state of a composition disclosed herein refers to the physical state of the composition as being a semi-solid or solid.

As used herein, “solid” and “semi-solid” refers to the physical state of a composition that supports its own weight at standard temperature and pressure and has adequate viscosity or structure to not freely flow. Semi-solid materials may conform to the shape of a container under applied pressure.

As used herein, a “solid phase medium,” “solid phase base” “solid phase substrate” “solid phase test substrate” “solid phase testing substrate,” and the like refer to a non-liquid medium, device, system, or environment. In some aspects, the non-liquid medium may be substantially free of liquid or entirely free of liquid. In one example, the non-liquid medium can comprise or be a porous material or a material with a porous surface. In another example, the non-liquid medium can comprise or be a fibrous material or a material with a fibrous surface. In yet another example, the non-liquid medium can be a paper.

As used herein, a “non-discoloration additive” refers to an additive that minimizes or prevents a color change in the color of the solid phase medium from an original or starting color to a different color for reasons other than nucleotide amplification from a LAMP reaction taking place thereon or therein. For example, in one embodiment, such a color change can be minimized or reduced as compared to a color change that would take place without the non-discoloration additive present.

As used herein, “non-LAMP reaction produced discoloration” refers to any discoloration (e.g., change in color from an original color to another color) of the solid phase medium which is not the result of a nucleotide amplification from a LAMP reaction. In some examples, non-LAMP reaction produced discoloration can refer to discoloration of the solid phase medium resulting from one or more of: a volatile agent, a magnesium-interfering agent, an oxidizing agent, a pH change resulting from causes other than amplification from a LAMP reaction, drying, or combinations thereof.

As used herein, a “volatile agent” refers to an agent that includes a composition that has a high vapor pressure or a low boiling point. In one example, ammonium sulfate can be a volatile agent because the ammonia can volatilize and leave behind sulfuric acid. In one example, a composition, component, or element, can have a high vapor pressure when the composition is in a gas phase at a temperature of more than about 30° C. In one example, a composition can have a low boiling point when the composition forms is in a gas phase at a temperature of less than about 80° C.

As used herein, a “pH-interfering reagent” is a reagent that can affect the pH of a reaction, system, or environment for reasons other than amplification from a LAMP reaction. In one example, the ammonium ion can volatilize from ammonium sulfate, and the sulfate ion can react to form sulfuric acid and affect the pH of the reaction in the absence of amplification from the LAMP reaction.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EMBODIMENTS

Many molecular tests for pathogens (e.g., severe acute respiratory syndrome coronavirus 2 (SARS-CoV-2), the virus responsible for COVID-19) can be limited to the laboratory and thus have significant lag times (>24 hours) to provide a result, preventing their adoption in point-of-care settings. Despite several attempts at developing a point-of-care test for SARS-CoV-2, some limitations remain: i) scalability (the demand for testing is in the order of millions per week, but manufacturing new tests at that scale is difficult), ii) sample processing (many tests still use an extraction operation when using saliva), and iii) readability (molecular tests often use fluorescence and thus, a fluorescence reader to report the results).

The current testing methods can be overcome by using a point-of-care test using paper-based devices and reverse-transcription loop-mediated isothermal amplification (RT-LAMP) that report a color change in the presence of a pathogen (e.g., SARS-CoV-2) within 60 minutes using diluted saliva (e.g., 5% v/v in water) as the sample. RT-LAMP is a nucleic acid amplification technique conducted at a constant temperature with adequate diagnostic performance especially during the acute phase of infection. Since RT-LAMP can be conducted at a constant temperature, expensive thermal cycling equipment is not used. Additionally, existing colorimetric reporters for LAMP products do not use fluorescence readers. Consequently, this test is suitable for use in point-of-care settings and is amenable to rapid development and scale-up, making it appropriate for use in public health emergencies.

RT-LAMP can be implemented on microfluidic paper-based analytical devices (μPADs) to detect various pathogens (e.g., SARS-CoV-2) where image analysis can be performed using a portable electronic device to distinguish between positive and negative responses. In one example, a high-contrast RT-LAMP reaction on paper can provide a color change that can be visible to the naked eye. In addition, instead of using wax-printing—which would have precise alignment of printed areas and dispensing of reagents—polystyrene spacers can be used for preventing crosstalk between samples. The polystyrene spacers can be amenable to roll-to-roll fabrication for scale up of production.

Nucleic-acid-based COVID-19 diagnosis methods use pre-processing to provide results. As disclosed herein, on-paper colorimetric detection of SARS-CoV-2 can be performed with minimal pre-processing. The device can have a sensitivity and specificity that can detect SARS-CoV-2 on paper without pre-amplification. Other assays conducted in solution may not be as scalable during manufacturing as paper-based assays. Additionally, the assay disclosed herein uses a dilution operation that can be completed in seconds, whereas other assays use various operations such as treatment with protease, heat-inactivation, and/or RNA extraction to detect SARS-CoV-2 (operations completed in at least 10 minutes and using additional equipment).

### Sample Collection and Properties for LAMP Analysis

Saliva has various physical, chemical, and antibacterial properties that can cause difficulty in the context of a LAMP reaction. For example, in one physical property, saliva can dilute and remove organic acids from dental plaque that can interfere with a LAMP reaction. Some of the chemical properties—electrolytes and buffering molecules that minimize changes in pH—can also interfere with a LAMP reaction. The antibacterial agents in saliva, e.g., mucins, amylases, lysozyme, and peroxidase enzyme, also present challenges. For example, peroxidase enzyme can form free radical compounds in bacterial cells that can cause them to undergo apoptosis-like death. However, such a reaction can also provide an unstable redox environment that can complicate a LAMP reaction.

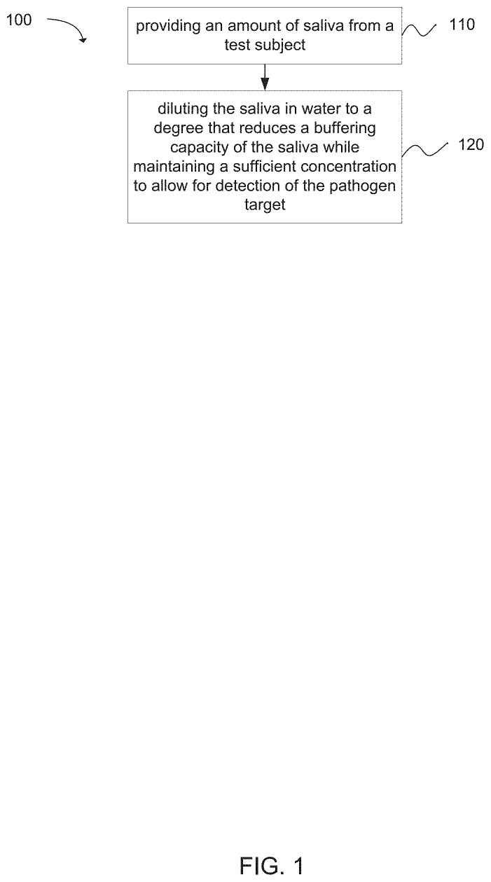

With the above-described background in mind, as depicted in the flowchart in FIG. 1, a method 100 of preparing a saliva sample for loop-mediated isothermal amplification (LAMP) detection of a pathogen target is provided. Depending on the final signal output selected to show a test result, for example an optically detected pH-based color change, it can be desirable to ensure that the saliva in the sample does not skew the overall sample pH significantly away from neutral. A variety of techniques and processes can be implemented to check or otherwise limit the buffering capacity or influence of the saliva in the sample. An excessive amount of buffering capacity can prevent the fluctuations in pH used to detect a pH-based color change.

One way of reducing the buffering capacity of saliva can include dilution. In one embodiment, such a method can comprise providing an amount of saliva from a test subject, as shown in block 110 and diluting the saliva in water to a degree that reduces a buffering capacity of the saliva while maintaining a sufficient concentration to allow for detection of the pathogen target, as shown in block 120.

The proteins present in saliva offer another challenge. For example, an excessively viscous sample can be difficult to test on a solid-based or solid-phase medium. A slow flow rate in a solid-based medium can increase the reaction time, decrease uniformity of spreading, increase variability in results, and increase invalidity of results. For example, when a viscous form of saliva does not spread evenly throughout a solid-based medium, a color-based indication can be difficult to read. The decrease in uniform spreading can also increase the variability of results by adding uncertainty to the reading of results. Different technicians may interpret the results differently. In some cases, the results may be impracticable to read due to ambiguous or absent color changes. Therefore, controlling the viscosity of the saliva can prevent various complications that may occur.

Therefore, in another embodiment, the method can further comprise reducing a viscosity of the saliva as compared to an original viscosity. In one aspect, the saliva can be reduced by one or more of dilution, filtering, the like, or combinations thereof. In one aspect, when the viscosity of the saliva is reduced using dilution, the saliva can be diluted in water to a saliva to water ratio of from about 1:1 to about 1:20. In another aspect, the viscosity of the saliva can be diluted in water to a saliva to water ratio of about 1:1, 1:2, 1:4, 1:8, 1:10, 1:12, 1:14, 1:16, 1:18, or 1:20. In one aspect, the saliva can be diluted in the water to a degree that provides the sample with an optical density at 600 nm (OD₆₀₀) of less than about 0.2. In one aspect, the saliva can have a volume range of from about 50 μl to about 100 μl. In another aspect, the saliva sample can have a volume ranging from about 100 μl to about 1 ml.

In some cases, diluting the saliva sample can reduce the impact of the effects arising from the buffering capacity and viscosity of the saliva. Another way of reducing the impact of the viscosity of the saliva can include filtering. In another aspect, the viscosity can be reduced using a filter having a rating between about 2 microns and 50 microns. In one example, the filter rating can be one or more of 2 microns, 5 microns, 8 microns, 10 microns, 15 microns, 20 microns, 25 microns, 40 microns, or 50 microns. In one aspect, the filter rating can be an absolute micron rating in which the filter can remove at least about 98.7% of a specific particle size. Filtering the saliva, rather than dilution alone, can also remove saliva proteins (e.g., mucins, amylases, lysozyme, and peroxidase enzyme) that may interfere with a LAMP reaction.

Through a combination of dilution, filtering, or both, the viscosity can be controlled to fall within a specific range. In yet another aspect, the viscosity can be reduced to a degree that increases flowability through a solid phase medium as compared to an original viscosity. In one example, a viscosity of saliva can have a range of from about 1 centipoise (cP) and to about 100 cP before dilution or filtering. In one example, the viscosity of the saliva can be reduced to a range of from about 1.0 cP to about 50 cP after dilution or filtering. In another example, the viscosity of the saliva can be reduced to a range of from about 1.0 cP to about 10 cP after dilution or filtering.

Filtering the saliva can also adjust the pH range to a desirable level. For example, some pH indicators may display a color change within a specific pH range (e.g., 7.2 to about 8.6). Therefore, the saliva can be filtered to a test sample target range depending on the type of pH indicator to be used. However, maintaining the test sample target range within physiological conditions can increase the uniformity of the LAMP reaction results. In one aspect, the saliva can be filtered to a degree that adjusts the saliva sample pH to a test sample target range. In one example, the test sample target range can include a pH range between about 7.2 to about 8.6. In another example, the test sample target range can include a pH range between about 7.6 to about 8.2.

Adjusting the test sample target range to a desired level may not be sufficient to detect a pH change (or other colorimetric indication) in a LAMP reaction. In another example, the saliva can be diluted with water to a degree that the buffering capacity of the composition is reduced relative to the buffering capacity before the dilution with water to allow a pH indication to be detected. In one example, buffering capacity can be defined as the ability of a solution (e.g., the saliva, the water, or the saliva diluted with water) to resist changes in pH when acids or bases are added. In one example, the buffering capacity can be defined as the amount of strong acid or strong base, grams equivalents, that is to be added to 1 liter of the solution to change the pH by one unit. In one aspect, the buffering capacity of the saliva can be between 0.03 mg/ml to about 0.30 mg/ml before dilution with water, and the buffering capacity of the saliva diluted water can be between about 0.003 mg/ml to about 0.03 mg/ml after dilution with water. In another example, the buffering capacity of the saliva diluted water can be less than about 5 mM, 4 mM, 3 mM, 2 mM, or 1 mM.

The water used to dilute the sample should be free of any contaminants or properties that might interfere with the LAMP reaction. For example, a pH that is too acidic can prevent the LAMP reaction from being detected if the pH prevents a pH-based indication change. In one aspect, the saliva can be diluted in water, wherein the water can have a pH greater than about 6.0. In another aspect, the water can have a pH less than about 8.0. In one example, the water can be molecular grade water that is substantially free of contaminants, such as RNase and DNase. RNase can degrade the RNA in the saliva that is to be detected, and DNase can degrade the DNA formed during the LAMP reaction. In another example, the saliva sample can consist essentially of saliva and water.

Minimizing the presence of undesirable saliva proteins can be accomplished using a specific saliva collection method. In one aspect, the saliva can be collected using one or more of a sponge-based collection method or a passive drool collection method. Collecting saliva using a sponge-based collection method may provide the benefit of inherently filtering mucins and high molecular weight proteins out of the saliva as they will not be absorbed by the sponge, which can reduce the viscosity of the saliva and increase the rapidity, uniformity, and reliability of the saliva when used on a solid-based medium. When the saliva is collected using a drooling method, the unfiltered saliva can have a greater viscosity, and therefore a reduced absorption, and distribution on a solid-based medium. As a result, in some embodiments when the saliva is collected via drooling, it can be subsequently filtered in order to remove mucins and other debris and reduce its viscosity.

A selected pathogen target can be detected from the saliva. In one aspect, the pathogen target can be one or more of a viral pathogen, a bacterial pathogen, a fungal pathogen, a protozoa pathogen, the like, or combinations thereof. The pathogen target in saliva can be detected when the nucleic acid from the pathogen target can be released from a cell wall, a cell membrane, a protein coat, or the like.

More specifically, in one aspect, the pathogen target can be a viral target. In some aspects, the viral target can be H1N1, H2N2, H3N2, H1N1pdm09, severe acute respiratory syndrome coronavirus 1 (SARS-CoV-1), severe acute respiratory syndrome coronavirus 2 (SARS-CoV-2), Middle East respiratory syndrome (MERS), influenza, the like, or combinations thereof.

The viral target can be selected from a number of different viral species. In one example, the viral target can be human coronavirus 229E, human coronavirus OC43, human coronavirus HKU1, human coronavirus NL63, MERS-coronavirus, human respirovirus 1, human rubulavirus 2, human respirovirus 3, human rubulavirus 4, human enterovirus, human respiratory virus, rhinovirus A, rhinovirus B, rhinovirus C, or combinations thereof.

The viral target can also be a form of influenza. In one aspect, influenza can be any of Influenza A, Influenza B, Influenza C, or Influenza D. In one aspect, the viral target can be a virus chosen from the order Nidovirale. In one aspect, the viral target can be chosen from the alpha, beta, gamma or delta genera of the Nidovirale order.

There are various families of viruses that may be detected. In one aspect, the viral target can be a DNA virus selected from the group of families including: Adenoviridae, Papovaviridae, Parvoviridae, Herpesviridae, Poxviridae, Anelloviridae, Pleolipoviridae, the like, and combinations thereof. In another aspect, the viral target can be an RNA virus selected from the group of families including: Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae, the like and combinations thereof. In another aspect, the viral target can be a reverse transcribing virus selected from the group of families including: Retroviridae, Caulimoviridae, Hepadnaviridae, the like, and combinations thereof.

More generally, the viral target can be a virus categorized by the Baltimore classification. In one aspect, the viral target can be an RNA virus (e.g., Influenza A, Zika, Hepatitis C). In one aspect, the viral target can be a DNA virus (e.g., Epstein Barr, Smallpox). In one aspect, the viral target can be a positive sense RNA virus (e.g., Hepatitis A, rubella). In one aspect, the viral target can be a negative sense RNA virus (e.g., Ebola, measles, mumps). In another aspect, the viral target can be a dsDNA virus (e.g., chickenpox, herpes), an ssDNA virus, a dsRNA virus (e.g., a rotavirus), a positive-strand ssRNA virus, a negative-strand ssRNA virus, an ssRNA-RT virus (e.g., retroviruses), or a ds-DNA-RT virus (e.g., Hepatitis B).

Besides viral targets, in another aspect, the pathogen target can be a bacterial target. In some examples, the bacterial target can be selected from a genus including: Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, Yersinia, the like, and combinations thereof. In another example, the bacterial target can be selected from a species including: Actinomyces israelii, Bacillus anthracis, Bordetella pertussis, B. abortus, B. canis, B. melitensis, B. suis, Corynebacterium diphtherias, E. coli, Enterotoxigenic E. coli, Enteropathogenic E. coli, Enteroinvasive E. coli, Haemophilus influenzae, Helicobacter pylori, Klebsiella pneumoniae, Legionella pneumophila, M tuberculosis, Mycoplasma pneumoniae, N. meningitidis, S. typhi, S. sonnei, S. dysenteriae, Streptococcus pneumoniae, Streptococcus pyogenes, Streptococcus viridans, Vibrio cholerae, Yersinia pestis, the like, and combinations thereof. In another aspect, the pathogen target can be selected from the species including: Chlamydia pneumoniae, Pneumocystis jirovecii, Candida albicans, Pseudomonas aeruginosa, Staphylococcus epidermis, Streptococcus salivarius, the like, and combinations thereof.

The pathogen target can also include various types of fungus. In one aspect, the pathogen target can be a fungal target. In some examples, the fungal target can be selected from a genus including: Aspergillus, Histoplasma, Pneumocystis, Stachybotrys, the like, and combinations thereof. In another aspect, the pathogen target can be a protist target. In some examples, the protist target can be selected from a genus including: plasmodium, trypanosomes, the like, and combinations thereof.

When the pathogen target in saliva includes RNA, the RNA can be reverse transcribed. Therefore, in another aspect, the LAMP detection can be reverse transcription LAMP (RT-LAMP). In this example, cDNA can be generated from a target RNA with a reverse transcriptase enzyme. The cDNA can be amplified to a detectable amount. When the pathogen target can be detected directly from DNA, then LAMP can be used to amplify the DNA to a detectable amount without reversed transcribing the RNA to DNA.

In another aspect, the specific target nucleotide sequences to be detected can be target nucleotides corresponding to human biomarkers. Any disease that has a target nucleotide corresponding to a human biomarker for a disease can be detected. Various types of diseases can be detected including one or more of: breast cancer, pancreatic cancer, colorectal cancer, ovarian cancer, gastrointestinal cancer, cervix cancer, lung cancer, bladder cancer, many types of carcinomas, salivary gland cancer, kidney cancer, liver cancer, lymphoma, leukemia, melanoma, prostate cancer, thyroid cancer, stomach cancer, the like, or combinations thereof. For example, biomarkers for various types of diseases can be detected by detecting target nucleotides corresponding to one or more of: alpha fetoprotein, CA15-3 and CA27-29, CA19-9, C!-125, calcitonin, calretinin, carcinoembryonic antigen, CD34, CD99MIC 2, CD117, chromogranin, chromosomes 3, 7, 17, and 9p21, cytokeratin, cesmin, epithelial membrane antigen, factor VIII, CD31 FL1, glial fibrillary acidic protein, gross cystic disease fluid protein, hPG80, HMB-45, human chorionic gonadotropin, immunoglobulin, inhibin, keratin, lymphocyte marker, MART-1, Myo D1, muscle-specific actin, neurofilament, neuron-specific enolase, placental alkaline phosphatase, prostate-specific antigen, PTPRC, S100 protein, smooth muscle action, synaptophysin, thymidine kinase, thyroglobulin, thyroid transcription factor-1, tumor M2-PK, vimentin, the like, or combinations thereof.

In another embodiment, a test sample composition for loop-mediated isothermal amplification (LAMP) analysis can comprise an amount of a test subject's saliva that is sufficient to detect a pathogen target via a LAMP analysis in combination with an amount of water that reduces a buffering capacity of the saliva. In one aspect, the viscosity of the composition can be from about 1.0 cP to about 50 cP. In another aspect, the pH of the composition can be from about 7.2 to about 8.6. Selecting a viscosity and pH within these respective ranges can enhance the change in pH and therefore the color change resulting from a pH-based indicator.

The saliva can be diluted with water to place the viscosity and pH within the ranges enumerated above. In one aspect, the saliva can be combined with the amount of water in a saliva to water ratio of from about 1:1 to about 1:20. In another aspect, the saliva to water ratio can be about 1:1, 1:2, 1:4, 1:6, 1:8, 1:10, 1:12, 1:14, 1:16, 1:18, or 1:20. In another aspect, the saliva can be combined with the amount of water to a degree that provides the sample with an optical density at 600 nm (OD₆₀₀) of less than 0.2.

In order to ensure that the amount of saliva contains a detectable amount of virus, the amount of collected saliva can be higher than a threshold amount. In one aspect, the saliva can have a volume ranging from about 50 μl to about 100 μl. In another aspect, the saliva sample can have a volume ranging from about 100 μl to about 1 ml.

The saliva can also have various chemical properties (e.g., pH and buffering capacity) that can facilitate the LAMP reaction. In one aspect, the water can have a pH greater than about 6.0 and can be substantially free of contaminants such as RNase and DNase. In another aspect, the water can have a pH less than about 8.0 and can be substantially free of contaminants. In another aspect, the composition can consist essentially of the saliva and the water. In one aspect, the buffering capacity of the composition can be between about 0.003 mg/ml to about 0.03 mg/ml. In another example, the buffering capacity of the composition can be less than about 5 mM, 4 mM, 3 mM, 2 mM, or 1 mM.

As disclosed in the preceding, the pathogen target can comprise a viral pathogen, a bacterial pathogen, a fungal pathogen, or a protozoa pathogen. The pathogen target can be a viral target. Based on the Baltimore classification of viruses, in another aspect, the viral target can comprise a dsDNA virus, an ssDNA virus, a dsRNA virus, a positive-strand ssRNA virus, a negative-strand ssRNA virus, an ssRNA-RT virus, or a ds-DNA-RT virus. In another aspect, the viral target can comprise H1N1, H2N2, H3N2, H1N1pdm09, or SARS-CoV-2.

### Reagent Compositions

A variety of reagents can be used in a LAMP analysis depending on the testing medium, readout type, and overall environment of the designed system. Further, reaction components such as primers and enzymes can be selected in view of the specific target nucleotide sequences to be detected, organisms to be identified, etc. Additionally, the specifics of the test environment, such as liquid environment, anhydrous environment, housing, substrates, etc. can be taken into account as well as other needs such as storage on stability when selecting specific reagents to be involved in the reaction underlying the LAMP analysis.

In one embodiment, a composition for loop-mediated isothermal amplification (LAMP) analysis on a solid phase medium can comprise one or more target primers, a DNA polymerase, and a re-solubilization agent. In one aspect, the composition can be substantially free of non-pH sensitive agents capable of discoloring the solid phase medium.

When conducting LAMP analysis on a solid phase medium, the concentration of reagents can be increased when compared to a LAMP analysis in a liquid phase medium. In one aspect, the concentration of DNA polymerase when used on the solid phase medium can be at least twice the concentration of DNA polymerase when used with a liquid medium. In another aspect, the concentration of DNA polymerase when used on the solid phase medium can be at least three times the concentration of DNA polymerase when used with a liquid medium. In one example, the concentration of DNA polymerase can be from about 300 U/mL to about 1000 U/mL when used on the solid-phase medium. In another example, the concentration of DNA polymerase can be from about 600 U/mL to about 1000 U/mL when used on the solid phase medium. In yet another example the concentration of DNA polymerase can be from about 620 U/m to about 680 U/mL when used on the solid phase medium.

When the LAMP analysis involves reverse transcriptase LAMP (RT-LAMP), the composition can further comprise reverse transcriptase. The reverse transcriptase can aid in the detection of RNA-based viruses. In one aspect, the concentration of reverse transcriptase when used on the solid phase medium can be at least twice the concentration of reverse transcriptase when used with a liquid medium. In another aspect, the concentration of reverse transcriptase can be least three times the concentration of reverse transcriptase when used with a liquid medium. In one example, the concentration of reverse transcriptase can be from about 200 U/mL to about 600 U/mL when used on the solid-phase medium. In another example, the concentration of reverse transcriptase can be from about 250 U/mL to about 500 U/mL when used on the solid phase medium. In yet another example the concentration of reverse transcriptase can be from about 290 U/mL to about 310 U/mL when used on the solid phase medium.

Besides the target primers, DNA polymerase, and reverse transcriptase, the composition can comprise a re-solubilization agent. A re-solubilization agent can aid in the re-hydration of the LAMP reagents on the solid-based medium when a saliva sample is deposited on the solid-based medium. In one aspect, the re-solubilization agent can be a surfactant. For example, the re-solubilization agent can comprise bovine serum albumin (BSA), casein, polysorbate 20, the like, or combinations thereof. BSA and casein can facilitate re-solubilization of the DNA polymerase, reverse transcriptase, and other related enzymes when the dried reagents are rehydrated. Polysorbate 20 is a surfactant that can also aid in the re-solubilization of dried reagents. In one example, the concentration of the re-solubilization agent can be from about 0.05 wt % to about 5 wt % when used on the solid-phase medium. In another example, the concentration of the re-solubilization agent can be from about 0.5 wt % to about 3 wt %. In yet another example, the concentration of the re-solubilization agent can be from about 0.5 wt % to about 1.5 wt %.

The composition can further comprise an agent that can speed up the reaction, increase sensitivity, or a combination thereof. In one example, BSA can be included to speed up the reaction and increase sensitivity. However, the inclusion of BSA can also introduce pH variations that can interfere with the readability of the results. Therefore, in some examples, the re-solubilization agent can include casein, polysorbate 20, the like, or combinations thereof.

Volatile agents can interfere with the LAMP reaction. For example, a volatile compound can ionize to a plurality of ions, and one of the ions can have a low boiling point. When the ion with the low boiling point evaporates, the remaining ion can further react. Some of the further reactions can include redox reactions, acid-base reactions, or other reactions that can affect the interpretation of a pH-based signal. In one aspect, the composition can be substantially free of volatile agents. In one example, the removal of volatile agents can increase the color contrast and decrease the reaction time of the solid-based medium when compared to the color contrast and reaction time when volatile agents are included. In one aspect, the composition can contain less than one or more of: 1.0 wt %, 0.5 wt %, 0.1 wt %, or 0.01 wt % of the volatile agents.

Volatile agents can cause instability in the solid-based medium. In some examples, a LAMP reaction that contains a volatile compound, such as ammonium sulfate, can cause instability in the solid-based medium when the ammonium ions partially convert the ammonium sulfate to ammonium which can volatilize and leave behind sulfate. The sulfate can become sulfuric acid and reduce the pH which can affect the reading of the pH-based indicator (e.g., changing the phenol red indicator from red to yellow even when the LAMP reaction does not occur). Replacing ammonium sulfate with betaine can prevent the non-LAMP reaction-based discoloration and stabilize the solid-based medium by preventing discoloration under storage.

As such, reducing the presence of volatile agents in the composition can reduce the degree of interference with the LAMP reaction and its reading via the pH-based indicator. In one aspect, the LAMP composition can comprise a non-volatile agent including a quaternary ammonium of low molecular weight of neutral charge, or an amide compound of low molecular weight of neutral charge, the like, or combinations thereof. In one example, the non-volatile agent can include, but is not limited to, N-Formylurea, Urea, L-Asparagine, Trimethylglycine (Betaine), 3-(Cyclohexylamino)-1-propanesulphonic acid (CAPS), 3-(1-Pyridinio)-1-propanesulfonate (NDSB-201), N-Methylurea, Acetamide, Propionamide, Isobutyramide, Piracetam, 1,3-Dimethylurea, 1,1-Dimethylurea, Glycolamide, 2-Chloroacetamide, Succinimide, 2-Imidazolidone, Choline chloride, Acetylcholine chloride, Bethanechol chloride, L-Carnitine inner salt, O-Acetyl-L-carnitine hydrochloride, 4-(Cyclohexylamino)-1-butanesulfonic acid (CABS), Dimethylethylammoniumpropane sulfonate (NDSB-195), 3-(1-Methylpiperidinium)-1-propane sulfonate (NDSB-221), 3-(Benzyldimethylammonio)propanesulfonate (NDSB-256), and Dimethyl-2-hydroxyethylammonium-1-propane sulfonate (NDSB-211), the like, or combinations thereof.

In one example, the concentration of the non-volatile agent including a quaternary ammonium of low molecular weight of neutral charge, or the amide compound of low molecular weight of neutral charge can be from about 1 mM to about 200 mM when used on the solid-phase medium. In another example, the concentration of the non-volatile agent can be from about 10 mM to about 50 mM when used on the solid-phase medium. In yet another example, the concentration of the non-volatile agent can be from about 15 mM to about 25 mM when used on the solid-phase medium.

In addition to volatile agents, hygroscopic agents can interfere with the LAMP reaction. A hygroscopic agent can retain an excessive amount of water and destabilize the reagents in the solid-based medium by slowing down or preventing drying. In one aspect, the composition can be substantially free of hygroscopic agents. In some examples, a LAMP reaction that contains a hygroscopic agent, such as glycerol can contribute to the instability of reagents in the solid-based medium because the hygroscopic agent attract can attract water. In one example, a hygroscopic agent can absorb more than about 10 wt % when between about 40% and about 90% relative humidity (RH) at 25° C. In one example, a hygroscopic agent can include, but is not limited to, one or more of glycerol, ethanol, methanol, calcium chloride, potassium chloride, calcium sulfate, the like, or combinations thereof. In one aspect, the composition can contain less than one or more of: 1.0 wt %, 0.5 wt %, 0.1 wt %, or 0.01 wt % of the hygroscopic agents.

Some additional agents can be included to prevent carryover contamination of previous LAMP reactions, primer dimerization, non-specific amplification, or a combination thereof. Carryover contamination can be prevented by including deoxyuridine triphosphate (dUTP), uracil DNA glycosylase (UDG), or a combination thereof in the LAMP reaction. These agents can catalyze the release of free uracil from single-stranded or double stranded DNA containing uracil.

It has been discovered that some pH-based indicators with an antioxidant effect, such as phenol red can have increased contrast and uniformity in comparison to other pH-based indicators with a reduced degree of antioxidant activity. In one aspect, the composition can further comprise an antioxidant. In one example, the concentration of the antioxidant can be from about 0.1 mM to about 1 mM when used on the solid-phase medium. In another example, the concentration of the antioxidant can be from about 0.2 mM to about 0.8 mM when used on the solid-phase medium. In yet another example, the concentration of the antioxidant can be from about 0.2 mM to about 0.3 mM when used on the solid-phase medium. The antioxidant can stabilize the reagents on the solid-based medium by preventing oxidization-reduction reactions.

Various antioxidants can be used including, but not limited to: N-acetyl-cysteine, hydroxytyrosol (HXT), superoxide dismutase (SOD), catalase, Vitamin A, Vitamin C, Vitamin E, coenzyme Q10, manganese, iodide, melatonin, alpha-carotene, astaxanthin, beta-carotene, canthaxanthin, cryptoxanthin, lutein, lycopene, zeaxanthin, apigenin, luteolin, tangeritin, isorhamnetin, kaempferol, myricetin, proanthocyanidins, quercetin, eriodyctiol, hesperetin, naringenin, catechin, gallocatechin, epicatechin, epigallocatechin, theaflavin, thearubigins, daidzein, genistein, glycitein, resveratrol, pterostilbene, cyanidin, delphinidin, malvidin, pelargonidin, peonidin, petunidin, chicoric acid, cholorogenic acid, cinnamic acid, ellagic acid, ellagitannins, gallic acid, gallotannins, rosmarinic acid, salicylic acid, curcumin, flavonolignans, xanthones, eugenol, capsaicin, bilirubin, citric acid, oxalic acid, phytic acid, R-alpha-Lipoic acid, the like, or combinations thereof.

Although pH-based indicators have been discussed thus far, other indicators can also be used. In one aspect, the composition can further comprise an indicator. In one example, the indicator can be a pH-based indicator, such as phenol red, when used with a solid-based medium. Phenol red has antioxidant properties that some other dyes do not have. The phenol red molecule is a conjugated bond system that might also contribute antioxidant properties. In one example, the concentration of the indicator can be from about 0.1 mM to about 1 mM when used on the solid-phase medium. In another example, the concentration of the indicator can be from about 0.2 mM to about 0.8 mM when used on the solid-phase medium. In yet another example, the concentration of the indicator can be from about 0.2 mM to about 0.3 mM when used on the solid-phase medium.

Some other indicators can also provide an adequate colorimetric signal. In another example, the indicator can be one or more of a (i) magnesium colorimetric indicator, (ii) a pH colorimetric indicator, or (iii) a DNA intercalating colorimetric indicator. When the indicator is a magnesium colorimetric indicator, the concentration of magnesium should be monitored to maintain the magnesium within a range of from about 0.01 mM to about 2 mM. Also, the concentration of magnesium should be monitored to prevent interference with DNA polymerase. Magnesium—a cofactor of DNA polymerase, can interfere with DNA polymerase when the magnesium concentration is outside a target range.

The LAMP reaction can also use various types of target primers. Some target primers can include about 4 or 6 primers that can target 6 or 8 regions within a genome, respectively. In one aspect, the concentration of the target primers can have a concentration from about 0.05 μM to about 5 μM when used on the solid-phase medium. In another example, the concentration of the target primers can be from about 0.1 μM to about 3 μM when used on the solid-phase medium. In yet another example, the concentration of the target primers can be from about 0.2 μM to about 1.6 μM when used on the solid-phase medium.

The target primers can be selected to target the genomes of various pathogens. In one aspect, the target primers can target a pathogen that can comprise a viral pathogen, a bacterial pathogen, a fungal pathogen, or a protozoa pathogen. In another aspect, the pathogen target can be a viral target. In another aspect, the viral target can comprise a dsDNA virus, an ssDNA virus, a dsRNA virus, a positive-strand ssRNA virus, a negative-strand ssRNA virus, an ssRNA-RT virus, or a ds-DNA-RT virus. In another aspect, the viral target can comprise H1N1, H2N2, H3N2, H1N1pdm09, or SARS-CoV-2. In sum, the target primers can target nearly any pathogen target, in particular, those target pathogens as disclosed herein.

When the solid-based medium includes an excessive amount of volatile agents, oxidizing agents, pH-interfering agents, magnesium-interfering agents, the like, or combinations thereof, then the color of the solid-based medium can be affected in the absence of amplification from the LAMP reaction. To address this issue, in another aspect, the composition can comprise a non-discoloration additive. In one aspect, the concentration of the non-discoloration additive can be from about 0.01 mM to about 1 M when used on the solid-phase medium. In another example, the concentration of the non-discoloration additive can be from about 10 mM to about 500 mM when used on the solid-phase medium. In yet another example, the concentration of the non-discoloration additive can be from about 200 mM to about 400 mM when used on the solid-phase medium.

There are various non-discoloration additives that can preserve the color of the solid-based medium in the absence of LAMP-reaction produced amplification and potentially increase the contrast when LAMP-reaction produced amplification occurs. In one example, the non-discoloration additive can comprise one or more of a sugar, a buffer, the like, or combinations thereof.

In one example, a non-discoloration additive such as sugar can stabilize the solid-based medium and prevent discoloration under long-term storage conditions. For example, trehalose can preserve the stability of enzymes under freeze-drying conditions or when dried at ambient temperatures. In one aspect, the sugar can comprise one or more of: glucose, sucrose, trehalose, dextran, the like, or combinations thereof. In one aspect, the concentration of the sugar can be from about 0.01 mM to about 1 M when used on the solid-phase medium. In another example, the concentration of the sugar can be from about 10 mM to about 500 mM when used on the solid-phase medium. In yet another example, the concentration of the sugar can be from about 200 mM to about 400 mM when used on the solid-phase medium.

The LAMP reaction can also include other reagents. In one aspect, the composition can comprise one or more of an enzyme, a nucleic acid, or combinations thereof. In one example, the enzyme can be an RNase inhibitor or a DNase inhibitor. Inclusion of an RNase inhibitor can slow the degradation of an RNA target to allow for an increased limit of detection. Inclusion of a DNase inhibitor can slow the degradation of a DNA target to also allow for an increased limit of detection. In one aspect, the composition can comprise carrier DNA or carrier RNA. The carrier DNA or carrier RNA can provide decoy substrate that sequesters the activity of DNase or RNase, respectively. In another example, a selected amount of guanidine hydrochloride can stimulate the denaturing and exposing of RNA molecules which can further stabilize the LAMP reaction.

In one aspect, the concentration of the RNase or DNase inhibitor can be from about 0.01 μL per mL of saliva sample to about 5 μL per mL of saliva sample when used on the solid-phase medium. In another example, the concentration of the RNase or DNase inhibitor can be from about 0.1 μL per mL of saliva sample to about 1 μL per mL of saliva sample when used on the solid-phase medium. In yet another example, the concentration of the RNase or DNase inhibitor can be from about 0.5 μL per mL of saliva sample to about 1.5 μL per mL of saliva sample when used on the solid-phase medium.

In one aspect, the concentration of the carrier RNA or carrier DNA can be from about 0.01 ng/μL to about 10 ng/μL when used on the solid-phase medium. In another example, the concentration of the carrier RNA or carrier DNA can be from about 0.1 ng/μL to about 1 ng/μL when used on the solid-phase medium. In yet another example, the concentration of the carrier RNA or carrier DNA can be from about 0.2 ng/μL to about 0.4 ng/μL when used on the solid-phase medium.

In addition to the foregoing, a number of other agents or ingredients can be used in a composition that is suitable for carrying out a LAMP reaction as recited herein. For example, in another aspect, the composition can further comprise a tonicity agent, a pH adjuster, a preservative, water, the like, or combinations thereof. Moreover, these ingredients/agents can be used to provide the composition with a range of specifically desired properties. In one aspect, the tonicity of the composition can be from about 250 to about 350 milliosmoles/liter (mOsm/L). In another aspect, the tonicity of the composition can be from about 270 to about 330 mOsm/L. Tonicity agents can be present in the composition in various amounts. In one aspect, the tonicity agent can have a concentration in the composition of from about 0.1 wt %, about 0.5 wt %, or about 1 wt % to about, 2 wt %, about 5 wt %, or about 10 wt %.

Although the composition should be substantially free of pH-interfering reagents, pH adjusters can be used to select an initial pH of the composition before a LAMP reaction. Furthermore, pH adjusters can also be used when the effects of the pH adjusters can be compensated for when interpreting results from the LAMP reaction. Non-limiting examples of pH adjusters can include a number of acids, bases, and combinations thereof, such as hydrochloric acid, phosphoric acid, citric acid, sodium hydroxide, potassium hydroxide, calcium hydroxide, and the like. The pH adjusters can be used to provide an appropriate pH for the composition. In one aspect, the pH can be from about 5.5 to about 8.5. In one aspect, the pH can be from about 5.8 to about 7.8. In another aspect, the pH can be from about 6.5 to about 7.8. In yet other examples, the pH can be from about 7.0 to about 7.6. pH adjusters can be present in the composition in various amounts. In one aspect, the pH adjuster can have a concentration in the composition of from about 0.01 wt %, about 0.05 wt %, about 0.1 wt %, or about 0.5 wt % to about 1 wt %, about 2 wt %, about 5 wt %, or about 10 wt %.

The shelf life of the composition can be enhanced by using preservatives. Non-limiting examples of preservatives can include benzalkonium chloride (BAK), cetrimonium, sodium perborate, ethylenediaminetetraacetic acid (EDTA) and its various salt forms, chlorobutanol, and the like. Preservatives can be present in the composition in various amounts. In one aspect, the preservative can have a concentration in the composition of from about 0.001 wt %, about 0.005 wt %, about 0.01 wt %, or about 0.05 wt % to about 0.1 wt %, about 0.25 wt %, about 0.5 wt %, or about 1 wt %.

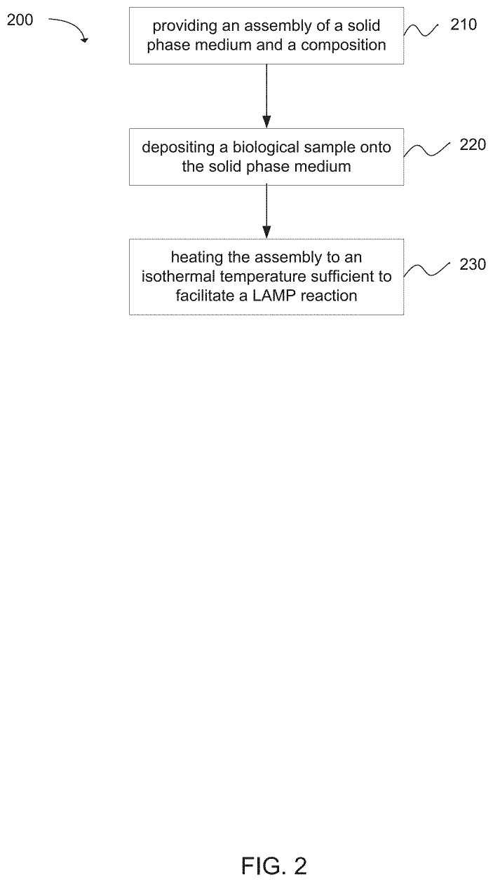

In another embodiment, as depicted in FIG. 2, a method 200 for LAMP analysis on a solid phase medium can comprise providing an assembly of a solid phase medium and a reaction composition in combination therewith, such as any of the ingredients or compositions as recited herein, as shown in block 210. In one aspect, the method can comprise depositing a biological sample onto the solid phase medium, as shown in block 220. In another aspect, the method can comprise heating the assembly to an isothermal temperature sufficient to facilitate a LAMP reaction, as shown in block 230.

In one aspect, the biological sample can be one or more of saliva, mucus, blood, urine, feces, sweat, exhaled breath condensate, the like, or combinations thereof. In another aspect, the biological sample can be saliva. In one aspect, the method can comprise detecting a viral pathogen. In one aspect, the viral pathogen can be a pathogen as disclosed herein. In another aspect, the LAMP analysis can be reverse transcriptase LAMP (RT-LAMP).

In another aspect, the isothermal temperature sufficient to facilitate a LAMP reaction can be in a temperature range from about 50° C. to about 70° C. In another aspect, the isothermal temperature sufficient to facilitate a LAMP reaction can be in a temperature range from about 60° C. to about 70° C. In another aspect, the isothermal temperature sufficient to facilitate a LAMP reaction can be in a temperature range from about 60° C. to about 65° C. The isothermal temperature can be selected based on one or more of the activity of the DNA polymerase, reverse transcriptase, or combinations thereof.

In another example, a temperature sufficient to facilitate a LAMP reaction can be in a temperature range of from about 60° C. to about 70° C. In another example, the isothermal temperature can be a temperature within a range that differs by less than 5 degrees Celsius.

In another embodiment, a system for performing a LAMP analysis can comprise a composition as recited in this disclosure. In another aspect, the system can comprise a solid phase medium on to which the composition is deposited.

Maximizing pH-Sensitive Signal Output

When conducting a LAMP reaction, various indicators can be used to read the results of the reaction. Three types of colorimetric indicators include magnesium colorimetric indicators, pH colorimetric indicators, and DNA intercalating colorimetric indicators. Because magnesium can be a cofactor for DNA polymerase and its concentration should be tightly controlled, magnesium-based indicators can face various limitations when used in the context of a LAMP reaction. DNA intercalating indicators can also face limitations because of the number of variables in play. Although all three indicators can be used in a LAMP Reaction, pH-based indicators may be subject to fewer variables.

In one embodiment, a composition for loop-mediated isothermal amplification (LAMP) analysis utilizing a pH-dependent output signal can comprise a pH sensitive dye, and a plurality of non-interfering LAMP reagents. In one aspect, the LAMP analysis can be reverse transcription LAMP (RT-LAMP).

The selection of pH-sensitive dye can depend on various factors, such as colorimetric range correlated to pH, degree of contrast between color changes, level of pH for a color change, uniformity of color change, reproducibility of color change, and the like. For example, phenol red can have a colorimetric range between a pH of about 6.8 and about 7.4. Below a pH of about 6.8, phenol red can turn yellow and above a pH of about 7.4, phenol red can turn red. The degree of difference between yellow and red can be simple to read, and the pH change can occur at a pH level that mimics physiological conditions.

In one aspect, the pH sensitive dye can be a pH indicator with a color change around a pH of 6.5 to achieve a consistent and contrasting color change (e.g., Phenol red). In one aspect, the pH sensitive dye can be at least one of phenol red, litmus, bromothymol blue, nitrazine yellow, cresol red, curcumin, brilliant yellow, m-cresol purple, α-naphtholphthalein, phenolphthalein, neutral red, acid fuchsin, azolitmin, the like, or combinations thereof. In one aspect, the concentration of the pH sensitive dye can be from about 0.1 mM to about 1 mM when used on the solid-phase medium. In another example, the concentration of the pH sensitive dye can be from about 0.2 mM to about 0.8 mM when used on the solid-phase medium. In yet another example, the concentration of the pH sensitive dye can be from about 0.2 mM to about 0.3 mM when used on the solid-phase medium.

To maximize the pH-sensitive signal output, the LAMP reaction should be substantially free of reagents that would introduce uncertainty into the signal by interfering with the LAMP reaction (e.g., interfering with the DNA polymerase) or by interfering with the signal from the LAMP reaction (e.g., the pH signal). In one aspect, the plurality of non-interfering LAMP reagents can comprise DNA polymerase, reverse transcriptase, target primers, or combinations thereof. In another aspect, the plurality of non-interfering LAMP reagents can be substantially free of volatile reagents, pH-interfering reagents, magnesium-interfering reagents, or combinations thereof.

In one example, the plurality of non-interfering LAMP reagents can be substantially free of magnesium, ammonium sulfate, or ammonium carbonate. Magnesium, as a cofactor of DNA polymerase, should be tightly monitored to ensure that the LAMP reaction can proceed as designed. Ammonium sulfate can ionize into the ammonium ion, which can leave behind a sulfate ion that can react to form sulfuric acid. Ammonium carbonate can also ionize into an ammonium ion and leave behind a carbonate that can react to form carbonic acid. Therefore, the plurality of non-interfering LAMP reagents should be substantially free of these substances.

Because volatile agents can leave behind a composition that can react to form an acid or base that can interfere with the pH-dependent signal from the LAMP reaction, volatile agents should be minimized. In one example, the plurality of non-interfering LAMP reagents can be substantially free of volatile reagents including, but not limited to: ammonium sulfate, and ammonium carbonate, the like, or combinations thereof. In one aspect, the composition can contain less than one or more of: 1.0 wt %, 0.5 wt %, 0.1 wt %, or 0.01 wt % of the volatile reagents.

Furthermore, any pH-interfering reagents can interfere with the pH-dependent signal output when the pH-interfering reagents is not compensated for. In one example, the plurality of non-interfering LAMP reagents can be substantially free of pH-interfering reagents including, but not limited to a number of acids, bases, and combinations thereof. In one aspect, the composition can contain less than one or more of: 1.0 wt %, 0.5 wt %, 0.1 wt %, or 0.01 wt % of the pH-interfering reagents.

Even when the pH has been monitored, the pH-dependent signal output can be negatively affected when the LAMP reaction is interfered with. For example, magnesium, as a cofactor of DNA polymerase, can interfere with the amplification from the LAMP reaction when the concentration is outside a selected range. In one example, the plurality of non-interfering LAMP reagents can be substantially free of magnesium-interfering agents. Magnesium-interfering agents can include magnesium-containing agents including, but not limited to: Mg²⁺, Mg¹⁺, magnesium carbonate, magnesium chloride, magnesium citrate, magnesium hydroxide, magnesium oxide, magnesium sulfate, magnesium sulfate heptahydrate, the like, or combinations thereof. In one aspect, the composition can contain less than one or more of: 1.0 wt %, 0.5 wt %, 0.1 wt %, or 0.01 wt % of magnesium. In another example, magnesium-interfering agents can include chelating agents that interfere with magnesium.

Even when the pH has been monitored and the LAMP reaction is functioning properly, discoloration of the solid-phase medium can result from other factors such as long-term storage. In one aspect, the composition can comprise a non-discoloration additive. In one example, the non-discoloration additive can comprise one or more of a sugar, a buffer, a blocking agent, the like, or combinations thereof. In one example, the sugar can stabilize the solid-based medium and prevent discoloration under long-term storage conditions. In one aspect, the sugar can comprise one or more of: glucose, sucrose, trehalose, dextran, the like, or combinations thereof.

In one aspect, the concentration of the sugar can be from about 0.01 mM to about 1 M when used on the solid-phase medium. In another example, the concentration of the sugar can be from about 10 mM to about 500 mM when used on the solid-phase medium. In yet another example, the concentration of the sugar can be from about 200 mM to about 400 mM when used on the solid-phase medium.

A buffer can facilitate the stabilization of the LAMP reaction by removing the variability from the saliva sample. In one example, a buffer can include one or more of phosphate-buffered saline (PBS), Dulbecco's PBS, Alsever's solution, Tris-buffered saline (TBS), HEPES, BICINE, water, balanced salt solutions (BSS), such as Hank's BSS, Earle's BSS, Grey's BSS, Puck's BSS, Simm's BSS, Tyrode's BSS, BSS Plus, Ringer's lactate solution, normal saline (i.e. 0.9% saline), ½ normal saline, the like, or combinations thereof. In one aspect, the concentration of the buffer can be from about 10 μM to about 20 mM when used on the solid-phase medium. In another example, the concentration of the buffer can be from about 100 μM to about 10 mM when used on the solid-phase medium. In yet another example, the concentration of the buffer can be from about 100 μM to about 500 μM when used on the solid-phase medium.

A blocking agent can decrease the amount of RNase-based degradation, DNase-based degradation, or other enzymatic degradations. In one example, a blocking agent can include one or more of bovine serum albumin, casein, or combinations thereof. In one aspect, the concentration of the blocking agent can be from about 0.01 wt % to about 5 wt % when used on the solid-phase medium. In another example, the concentration of the blocking agent can be from about 0.01 wt % to about 1 wt % when used on the solid-phase medium. In yet another example, the concentration of the blocking agent can be from about 0.02 wt % to about 0.06 wt % when used on the solid-phase medium.

An antioxidant can increase the uniformity and contrast of the pH-dependent signal on the solid-phase medium by eliminating variables associated with oxidation reactions. In one example, the composition can further comprise an antioxidant as disclosed herein.

In another example, the composition can further comprise a solid phase medium. The solid-phase medium can include, but is not limited to, one or more of: glass fiber, nylon, cellulose, polysulfone, polyethersulfone, cellulose acetate, nitrocellulose, polyester, hydrophilic polytetrafluoroethylene (PTFE), or combinations thereof.

Certain additives may increase the stability and uniformity of the LAMP reaction. In one aspect, the composition can comprise one or more of an enzyme, a nucleic acid, or combinations thereof as disclosed herein. In one example, the enzyme can be an RNase inhibitor or a DNase inhibitor. In another aspect, the composition can comprise carrier DNA or carrier RNA. The carrier DNA or carrier RNA can provide decoy substrate that sequesters the activity of DNase or RNase, respectively.

In another example, a selected amount of guanidine hydrochloride can stimulate the denaturing and exposing of RNA molecules. In one aspect, the concentration of the guanidine hydrochloride can be from about 1 mM to about 200 mM when used on the solid-phase medium. In another example, the concentration of the guanidine hydrochloride can be from about 10 mM to about 100 mM when used on the solid-phase medium. In yet another example, the concentration of the guanidine hydrochloride can be from about 20 mM to about 60 mM when used on the solid-phase medium.

The maximization of the pH-dependent output signal can also be used in conjunction with other embodiments as disclosed herein. In one embodiment, a method of performing a LAMP analysis with a pH-dependent output signal can comprise providing an assembly of a solid phase medium and a composition as recited herein. The method can further comprise depositing a biological sample onto the solid phase medium. The method can further comprise heating the assembly to an isothermal temperature sufficient to facilitate a LAMP reaction.

As disclosed herein, in one aspect, the biological sample can be one or more of saliva, mucus, blood, urine, feces, sweat, exhaled breath condensate, the like, or combinations thereof. In another aspect, the biological sample can be saliva. In one aspect, the method can comprise detecting a viral pathogen. In one aspect, the viral pathogen can be a pathogen as otherwise disclosed herein.

In one example, a temperature sufficient to facilitate a LAMP reaction can be in a temperature range of from about 60° C. to about 70° C. In another example, the isothermal temperature can be a temperature within a range that differs by less than 5 degrees Celsius.

In another aspect, the isothermal temperature sufficient to facilitate a LAMP reaction can be in a temperature range from about 50° C. to about 70° C. In another aspect, the isothermal temperature sufficient to facilitate a LAMP reaction can be in a temperature range from about 60° C. to about 70° C. In another aspect, the isothermal temperature sufficient to facilitate a LAMP reaction can be in a temperature range from about 60° C. to about 65° C. The isothermal temperature can be selected based on one or more of the activity of the DNA polymerase, reversed transcriptase, or combinations thereof.

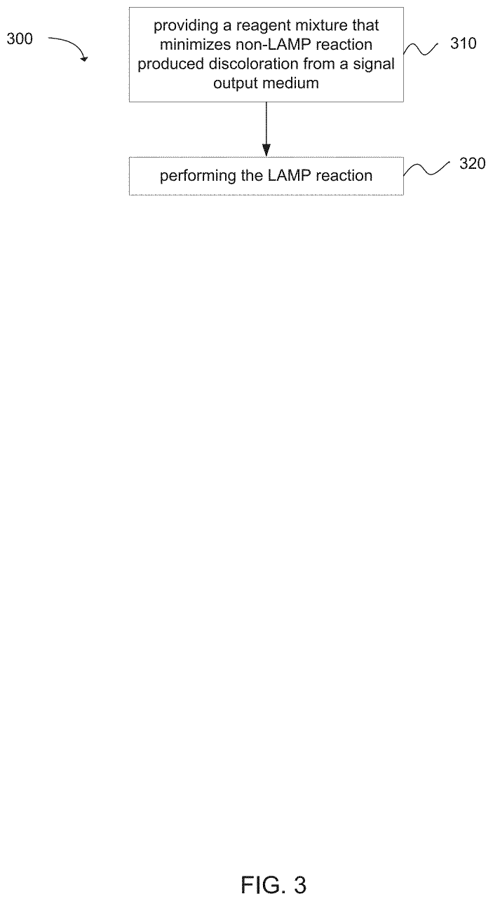

In another embodiment, as depicted in FIG. 3, a method 300 of maximizing accuracy of an output signal in a pH-dependent LAMP analysis can comprise providing a reagent mixture that minimizes non-LAMP reaction produced discoloration from a signal output medium as in block 310. The method can further comprise performing the LAMP reaction, as shown in block 320. In one aspect, the method can comprise controlling production of protons from a non-LAMP reaction. In another aspect, the method can comprise controlling oxidation from a non-LAMP reaction.

In another embodiment, a method of maximizing accuracy of an output signal in a pH-dependent LAMP analysis can comprise substantially eliminating non-LAMP reaction produced discoloration from a signal output medium.

In another embodiment, a method of maximizing a level of detection (LOD) in a pH-dependent LAMP analysis can comprise substantially eliminating non-LAMP reaction produced discoloration from a signal output medium. In one aspect, the color contrast can be enhanced and the sample variability can be mitigated without impacting the limit of detection by diluting the saliva to 5-10% with water. In another example, the color contrast can be enhanced and the sample variability can be mitigated without impacting the limit of detection by filtering the saliva with a filter as otherwise disclosed herein.

### EXAMPLES

The following examples are provided to promote a more clear understanding of certain embodiments of the present invention, and are in no way meant as a limitation thereon.

### Paper LAMP Analysis for Viral Targets in Diluted Saliva Samples

### Example 1—DNase/RNase-Free Distilled Water

DNase/RNase-free distilled water is prepared by filtration with 0.1 μm membrane and tested for DNase and RNase activity. The DNase and RNase activity is tested in accordance with current U.S. Pharmacopeia (USP) monograph test standards for Water for Injection (WFI). Upon confirmation of no DNase, RNase, or protease activity the water is considered contaminant free and ready for use in preparing saliva samples.

### Example 2—Amplification in Saliva

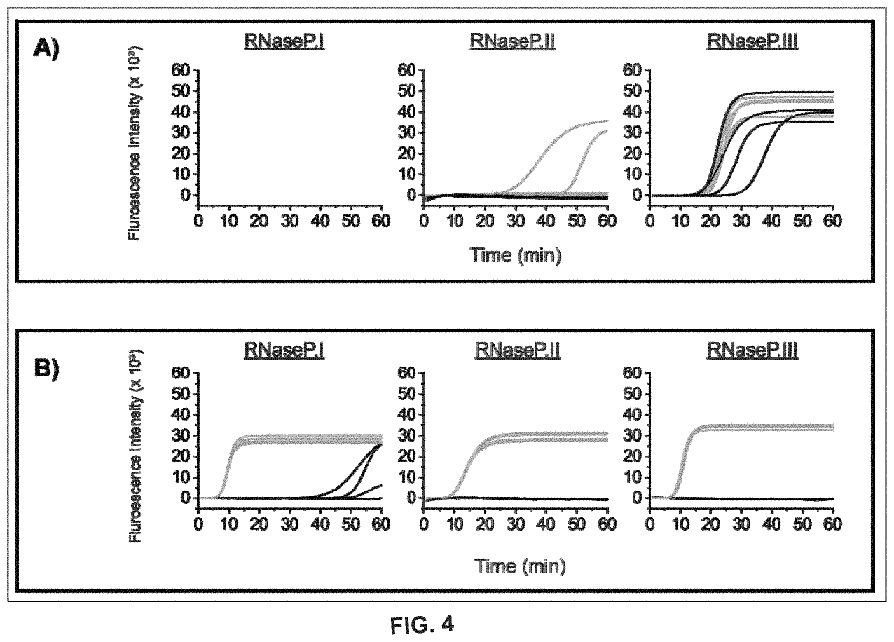

A nucleic acid sequence primer was designed to target RNaseP in saliva as a positive control to confirm nucleotide amplification from a saliva sample as illustrated in FIG. 4. FIG. 4A illustrates flouorometric RT-qLAMP results for primer sets targeting RNaseP POP7 in 18% saliva spiked with 105 genome equivalents/reaction of heat-inactivated SARS-CoV-2. FIG. 4B illustrates flouorometric RT-qLAMP results for primer sets targeting RNaseP POP7 in water with 0.2 ng of synthetic RNaseP POP7 RNA.

As illustrated in FIG. 4A, 18% saliva that has been spiked with 105 genome equivalents per reaction of heat-inactivated SARS-CoV-2 was analyzed. In the left figure, no amplification occurred because the primer (RNaseP.I) which was designed to target RNaseP using the mRNA sequence for the POP7 gene which encodes for the p20 subunit of RNaseP, did not adequately detect the low levels of the RNaseP. In the middle figure, amplification occurred, as shown in the blue lines without overlap with the black lines, because the primer (RNaseP.II) was able to detect levels of RNaseP without amplifying the no template control. In the right figure, amplification occurred for both the black lines and the blue lines because the primer (RNaseP.III) dimerized (e.g., showed amplification in the no template control black lines).

As illustrated in FIG. 4B, water with 0.2 ng of synthetic RNaseP POP7 RNA was analyzed. In the left figure, amplification occurred in the blue lines and black lines because the primer (RNaseP.I) dimerized (e.g., amplified the no template control). In the middle figure, amplification occurred, as shown in the blue lines without overlap with the black lines, because the primer (RNaseP.II) was a was able to detect the levels of the RNaseP without amplifying the no template control. In the right figure, amplification occurred for the blue lines but not the black lines because the primer (RNaseP.III) amplified the RNase P without amplifying the no template control.

### Example 3—Saliva Collection Devices

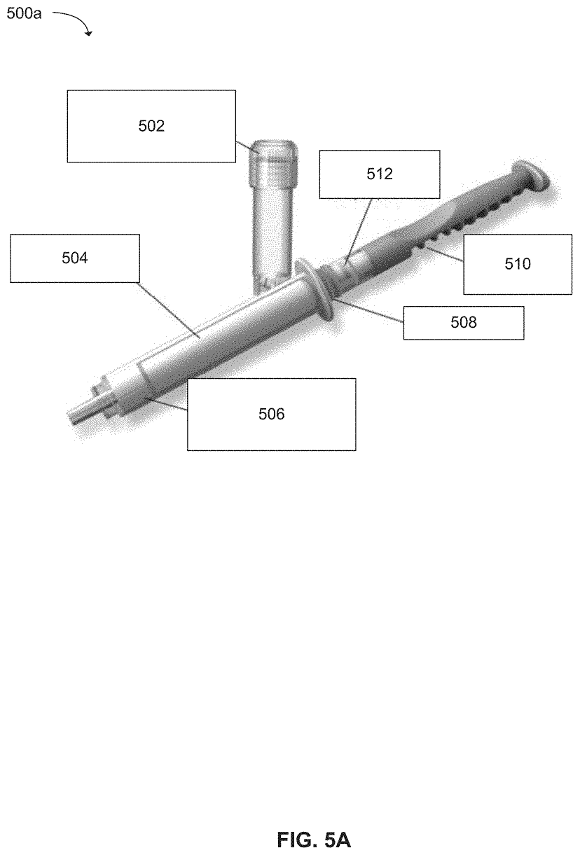

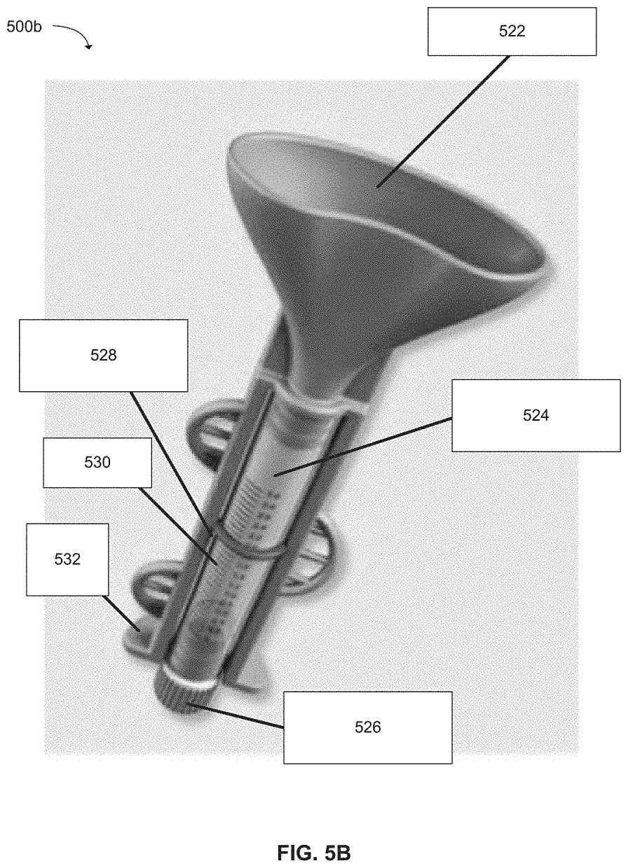

The type of saliva collection device can facilitate a saliva sample in a LAMP reaction. In some cases, an operator may use protective equipment to protect from a pathogen that can be spread via airborne droplets (e.g., an aerosol virus). Therefore, operators can wear personal protective equipment to protect against the accidental contact with the aerosol virus. The specific saliva collection device may be self-administered by the subject under the guidance of a healthcare professional. The saliva collection device has a proven efficacy and can fall into two categories: sponge-based collection and passive drool collection as illustrated in FIGS. 5A and 5B.

A sponge-collection device 500a uses a sponge-like collection pad 504 to absorb saliva and includes a sample volume adequacy indicator 512 to indicate when sufficient volume has been collected. Once saturated, the sponge is inserted into a compression tube 506 and compressed against a filter which strains the saliva into a collection tube. A reason for this filtration operation is that it strains mucins and high molecular weight proteins out of the saliva and significantly reduces viscosity of the specimens. As a result, the solid phase medium can take-up and distribute the saliva in a more rapid, uniform, and reliable fashion. The sponge-collection device 500a can also include: a compression seal 508 on the compression tube 506 to form a seal with the compression tube; a handle 510 to compress the compression tube 506; and a sample volume adequacy indicator 512 to identify when sufficient saliva has been collected.

A passive drool device 500b can provide unfiltered saliva, with viscosity that slows absorption and distribution of the sample. The passive drool device 500b can include: a collection funnel 522 for collecting saliva; an indicator line 528 for indicating when sufficient saliva has been collected; a collection tube 524 to collect the saliva; a tube cap 526; a volume indicator 530; and a tube cap storage 532.

For both types of collection devices, residual risk of exposure to the operator is minimized. With the sponge-based devices, there is a hypothetical risk of aerosol release during the compression operation, especially if a user is particularly abrupt in the compression process. The healthcare operator may perform this operation to control the risk of exposure. The collection device may include a compression seal to prevent aerosol backflow. With passive-drool collection, there may be some risk of contaminating the outside of the device with stray saliva, which could then transmit a cross-contamination to the operator if not handled properly. In both cases, exposure risks are mitigated by the patient self-collection of the saliva sample.

Three commercial saliva collection devices were selected to evaluate their effect on the RT-LAMP reaction in saliva. The three devices were “Saliva Sampler™” produced by StatSure Diagnostic Systems, Inc., “Pure⋅SAL™” produced by Oasis Diagnostics, and “Super⋅SAL™” also produced by Oasis Diagnostics. The StatSure Saliva Sampler™ provides a tube containing a buffer (e.g., Buffer 2000) used to collect saliva from a patient. Super⋅SAL uses a cylindrical absorbent pad and a collection tube to standardize the collection of saliva by removing any solid contaminants and mucinous material. Pure⋅SAL operates on a similar mechanism but includes an additional filter in the collection tube to remove contaminants.

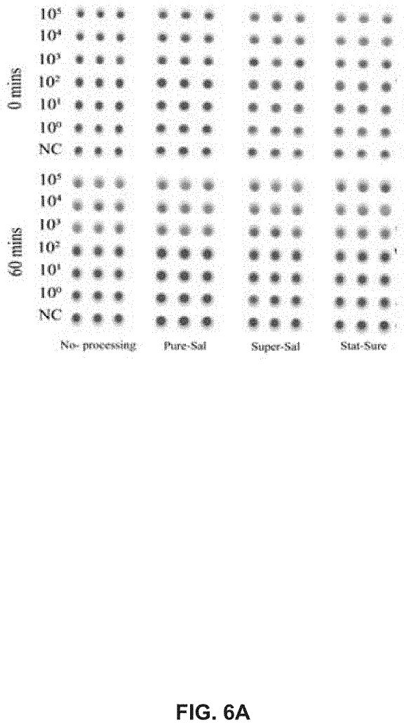

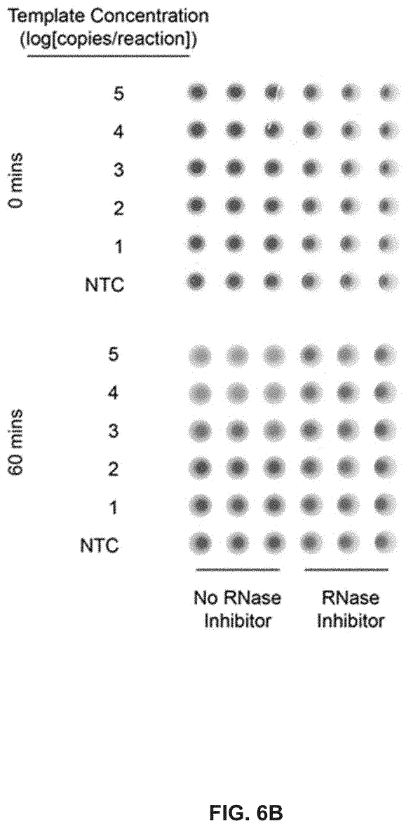

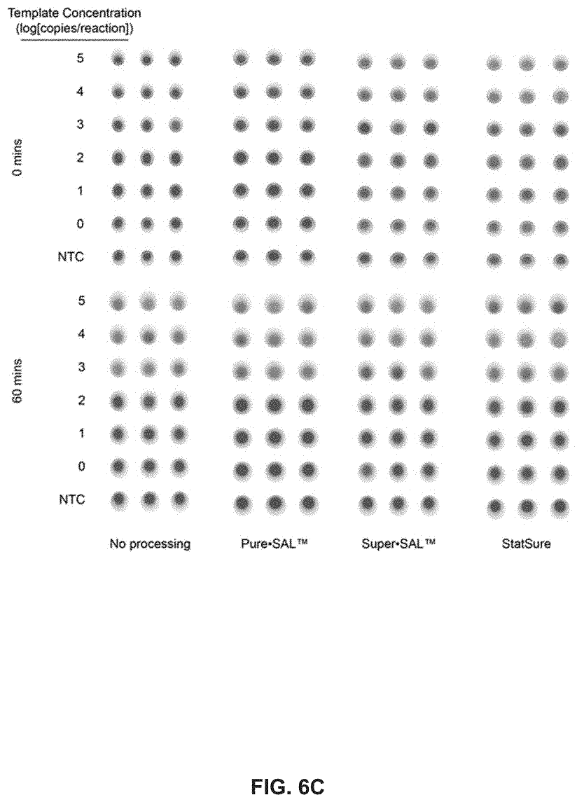

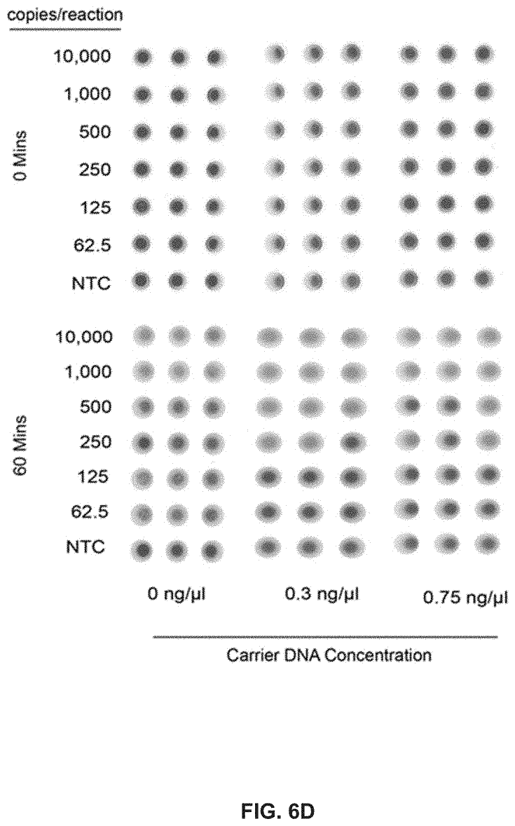

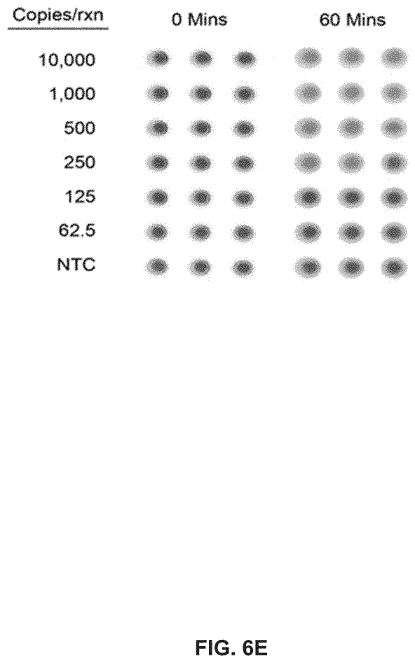

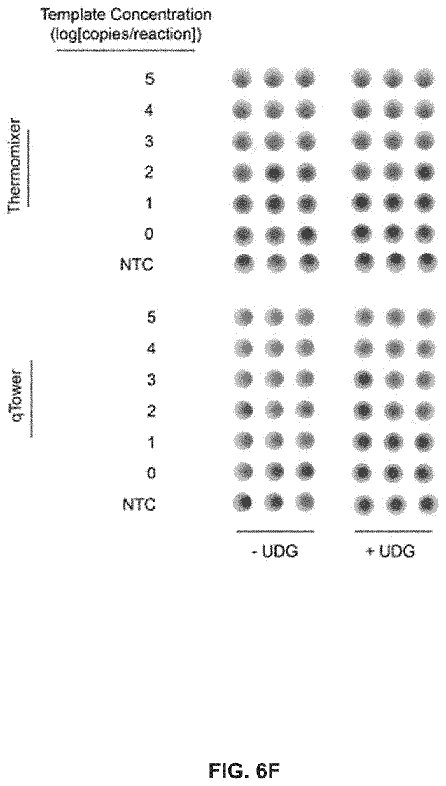

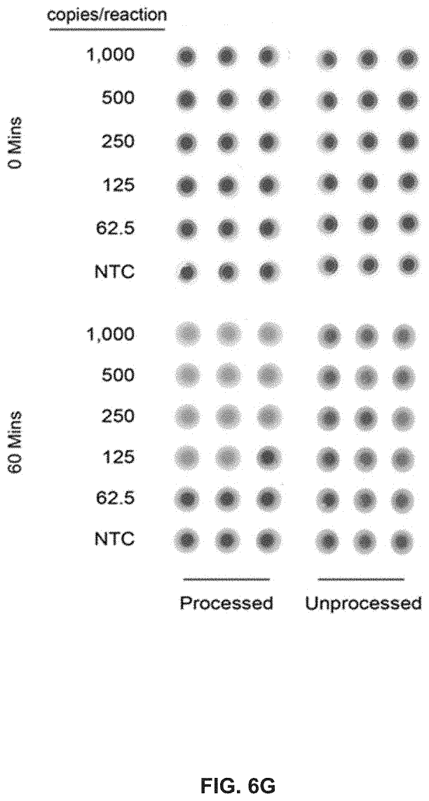

The saliva pH was measured from processed saliva samples, and subsequent colorimetric and fluorescent RT-LAMP LOD assays were run using processed saliva samples. This data is presented in FIG. 6A. This data illustrates the LoD in saliva processed using different saliva collection devices (Pure-Sal, Super-Sal, Stat-sure). The master-mix was treated with 0.6 microliter of HCl. The Pure⋅SAL™ and Super⋅SAL saliva collection devices illustrate a wider range of colorimetric response for a wider range of concentrations (1 to 10 k genome equivalents per reaction of heat inactivated SARS-CoV-2).

### Example 4—Saliva Collection Process

If a subject is self-testing, the subject collects a saliva specimen under the guidance of the healthcare professional into a specialized collection vessel which contains no additives and is thus safe for the subject to use in the collection. The collected volume of saliva is approximately 100 μL. The sponge sampler for example is inserted into the subject's mouth and saliva is collected until the indicator on the sponge sampler changes color. The sponge sampler is then inserted into a collection tube. The sponge is then compressed to squeeze out the saliva (approximately 100 μL) into a collection tube containing an amount of water in it to dilute the saliva. The saliva is diluted in the water to a saliva to water ratio of about 1:1 to about 1:20. The saliva is transferred from the collection tube to a test site.

### Example 5—RNase Inhibitor Effect On Saliva

An RNase inhibitor was added to untreated saliva at a concentration of 1 μL per mL of saliva to determine the effect of the addition of an RNase Inhibitor on the RT-LAMP reaction.

The effect of RNAsecure™ (AM7006, Invitrogen™) on freshly collected saliva (5%) was tested to determine its suitability as a single operation process for a point-of-care RT-LAMP reaction. 1×RNAsecure™ was diluted from 25× stock using 1 ml of saliva. The treated saliva was used as a matrix to spike heat-inactivated SARS-CoV-2 with a concentration range from 1000 copies to 62.5 copies/reaction into the Warmstart™ colorimetric master mix along with 40 mM of guanidine hydrochloride and 0.3 ng/μl carrier DNA with a pH of 7.6. The RNAsecure™ treated RT-LAMP was incubated at 65° C. to start the reaction. Fresh saliva under these conditions was tested without RNAsecure™ as a control.

5 μL of heat-inactivated virus was diluted in 5% processed saliva (i.e., final reaction concentration) and added to the RT-LAMP reaction to provide the indicated concentration for a final reaction volume of 25 μL. For negative reactions, 5 μL of processed saliva (5% final reaction concentration) was added in lieu of diluted heat-inactivated virus to provide the same reaction volume of 25 μL. Heating was conducted in the incubator at 65° C. for 60 mins. The colorimetric scans were taken using the flatbed scanner before and after the RT-LAMP reaction. 1250 μL of NEB colorimetric master mix was supplemented with 0.5 μL of Antarctic Thermolabile, 3.5 μL of dUTP. 25×. RNase Inhibitor (RNASecure™) was diluted in whole saliva to result in a 1× concentration prior to adding to the reaction diluting to 5% saliva.

RNAsecure™ did not show any significant increase in the LoD of the reaction, as illustrated in FIG. 6B. That is, the addition of RNase inhibitor did not result in an appreciable increase in the measured parameters of the RT-LAMP reaction (e.g., the reaction speed, the false-positive rate, or the limit of detection).

### Example 6—Frozen Saliva Samples

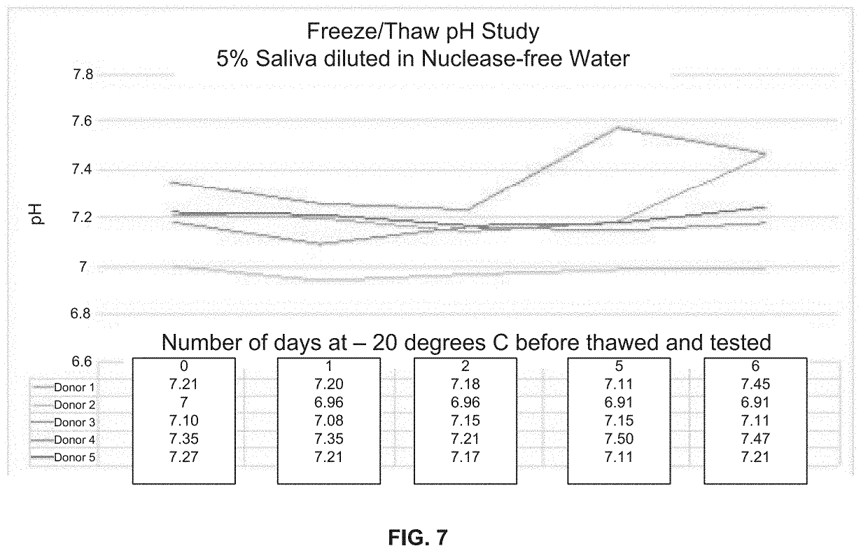

In some instances, it may be desirable to freeze a saliva sample for a period of time prior to its analysis due to logistics, need for transport, etc. Such situations may merit specific care when performing a LAMP analysis as recited herein. As illustrated in FIG. 7, the pH of a frozen saliva sample can vary depending on the number of days at −20 degrees C. before the saliva sample is thawed and tested. In one example, the pH of the saliva sample from Donor 1 varied from a pH of 7.21 without any days between collection and testing to a pH of 7.46 after 6 days between collection/freezing and testing. In another example, the pH of the saliva sample from Donor 2 varied from a pH of 7.00 without any days between collection and testing to a pH of 6.98 after 6 days between collection/freezing and testing. In one example, the pH of the saliva sample from Donor 3 varied from a pH of 7.18 without any days between collection and testing to a pH of 7.18 after 6 days between collection/freezing and testing. In one example, the pH of the saliva sample from Donor 4 varied from a pH of 7.35 without any days between collection and testing to a pH of 7.47 after 6 days between collection/freezing and testing. In one example, the pH of the saliva sample from Donor 1 varied from a pH of 7.22 without any days between collection and testing to a pH of 7.24 after 6 days between collection/freezing and testing.

### Example 7—Limit of Detection in Fresh Saliva

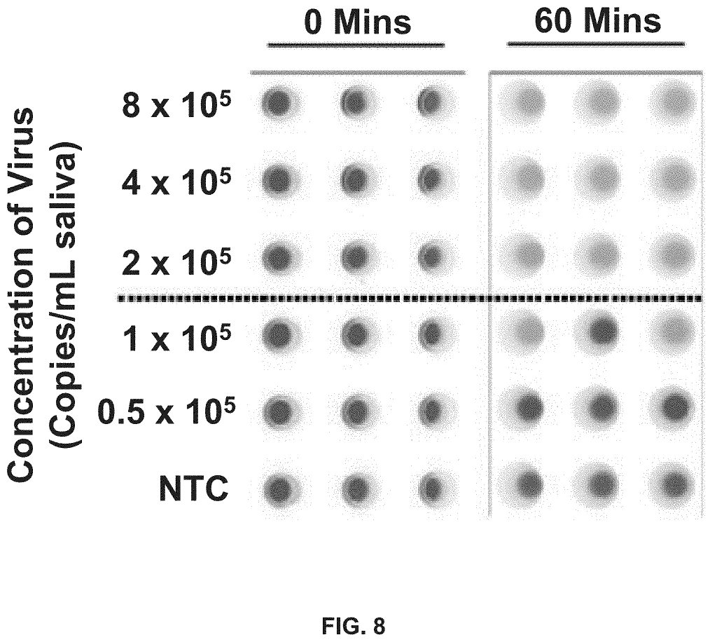

FIG. 8 illustrates the limit of detection of fresh saliva. Fresh saliva was collected using a drooling method and was diluted in water in a 1:3 ratio to obtain 25% saliva and 75% water. Heat-inactivated SARS-CoV-2 was spiked into the 25% saliva with serial dilutions, as a control. 5 μL of 25% saliva was added to 20 μL of RT-LAMP reagents so that the final concentration of saliva was 5%. After incubating at 65° C. for 1 hour the color changed. The number of copies on the y-axis represents what the original concentration of 100% saliva would have be without dilution. The limit of detection (LOD) for the primer was 250 copies/reaction in a volume of 25 μL, which is equivalent to about 200 k copies/mL of saliva.

Thus, it was determined that diluting the saliva to 25% with nuclease-free water and further diluting to a final concentration of 5% saliva upon addition to the RT-LAMP reaction could obtain results within 60 minutes. Dilution reduced the buffering capacity of saliva and decreased the concentration of inhibitory components, both of which would delay colorimetric reporting. Dilution is less complex to the end-user compared to other pre-treatment operations found in a variety of studies, such as pre-treatment with proteases, Chelex® 100, or RNA extraction operations to inactivate inhibitory components of saliva.

The LoD of the colorimetric assay in 5% saliva that has been processed using Pure⋅SAL™ is 1000 copies/reaction (reaction volume 25 μL), which corresponds to 800 copies/μL of patient saliva after accounting for dilution (FIG. 6C).

As illustrated in FIG. 6C, different saliva collection devices (Pure⋅SAL™, Super-Sal™, Stat-Sure™) can result in varying LoD. 5% diluted saliva with water was tested for all the processing techniques. Primer set orf1ab.2 was used. 5 μL of heat-inactivated virus diluted in 5% processed saliva (final reaction concentration) was added to the RT-LAMP reaction to result in the indicated concentration and a final reaction volume of 25 μL. For negative reactions, 5 of processed saliva (5% final reaction concentration) was added in lieu of diluted heat-inactivated virus to provide the same reaction volume of 25 μl. Heating was conducted in an incubator at 65° C. for 60 mins. The colorimetric scans were taken using the flatbed scanner before and after the RT-LAMP reaction. Reactions consisted of 12.5 μL of NEB 2× colorimetric master mix, 2.5 μL of primer mix, 5 μL of water, and 5 μL of sample.

This LoD is several orders of magnitude higher than RT-PCR assays or other assays utilizing RNA extraction (on the order of 1 copy/reaction). However, these other assays were accompanied by pretreatment protocols and/or RNA extraction operations to achieve the reported LoD.

To enhance this LoD, we investigated the use of RNase inhibitors, Guanidine HCl, and carrier DNA. The addition of RNase inhibitors lowered the LoD in 5% saliva (FIG. 6B), which contradicts literature reports in which RT-LAMP assays in saliva utilize RNase inhibitors increased the LoD; this discrepancy may be due to the type of RNase inhibitor used. Both Guanidine HCl and carrier DNA increased the LoD (FIG. 6D and FIG. 6E) and was added to our RT-LAMP reaction formulation for colorimetric solution reactions. These components were not included because of a color change when drying on paper.

As illustrated in FIG. 6D, 5 μL of heat-inactivated virus diluted in 5% processed saliva (final reaction concentration) was added to the RT-LAMP reaction to result in the indicated concentration and a final reaction volume of 25 μL. For negative reactions, 5 μL of processed saliva (5% final reaction concentration) was added in lieu of diluted heat-inactivated virus to result in the same reaction volume of 25 μl. Primer set orf1ab.2 was used. Heating was conducted in an incubator (Fisherbrand™ Isotemp™) at 65° C. for 60 mins. The colorimetric scans were taken using the flatbed scanner before and after the RT-LAMP reaction. 1250 μL of NEB colorimetric master mix was supplemented with 0.5 μL of Antarctic Thermolabile UDG, 3.5 μL of dUTP, and carrier DNA to result in a final reaction concentration as indicated.

As illustrated in FIG. 6E, 5 μL of heat-inactivated virus diluted in 5% processed saliva (final reaction concentration) was added to the RT-LAMP reaction to result in the indicated concentration and a final reaction volume of 25 μL. For negative reactions, 5 μL of processed saliva (5% final reaction concentration) was added in lieu of diluted heat-inactivated virus to result in the same reaction volume of 25 μl. Primer set orf1ab.2 was used. Heating was conducted in an incubator (Fisherbrand™ Isotemp™) at 65° C. for 60 mins. The colorimetric scans were taken using the flatbed scanner before and after the RT-LAMP reaction. 1250 μL of NEB colorimetric master mix was supplemented with 0.5 μL of Antarctic Thermolabile UDG, 3.5 μL of dUTP, and Guanidine HCl (40 mM).

Finally, uracil-DNA glycosylase (UDG) and deoxyuridine triphosphate (dUTP) (FIG. 6F) were included to reduce carryover contamination. As illustrated in FIG. 6F, Colorimetric scans after 60 minutes of incubation at 65° C. for 25 μL reactions on the thermomixer and incubator with and without the addition of UDGs and dUTP. The Primer set used was orf1ab.II. The template was heat-inactivated virus at the indicated concentration. For reactions with UDG, 1250 μL of NEB 2× colorimetric master mix was supplemented with 0.5 μL of Antarctic Thermolabile UDG, 3.5 μL of dUTP. For all other reactions NEB 2× colorimetric master mix was used.

When including Guanidine HCl, carrier DNA, and UDG, the LoD of the RT-LAMP colorimetric assay in 5% processed saliva in solution increased to 250 copies/reaction (FIG. 6G). As illustrated in FIG. 6G, RT-LAMP colorimetric LoD using saliva processed with Pure⋅SAL™ and saliva that was unprocessed. Plates were heated in an incubator set at 65° C. for 60 minutes. The Primer set used was orf1ab.II and the template was heat-inactivated virus at the indicated concentration (positive reactions) or nuclease-free water (negative reactions). Heating was conducted in an incubator (Fisherbrand™ Isotemp™) at 65° C. for 60 mins. The colorimetric scans were taken using the flatbed scanner before and after the RT-LAMP reaction. 1250 μL of NEB colorimetric master mix was supplemented with 0.5 μL of Antarctic Thermolabile UDG, 3.5 μL of dUTP, carrier DNA (0.3 ng/μL), and Guanidine HCl (40 mM).

### Example 8—Limit of Detection in Animal Nasal Swab

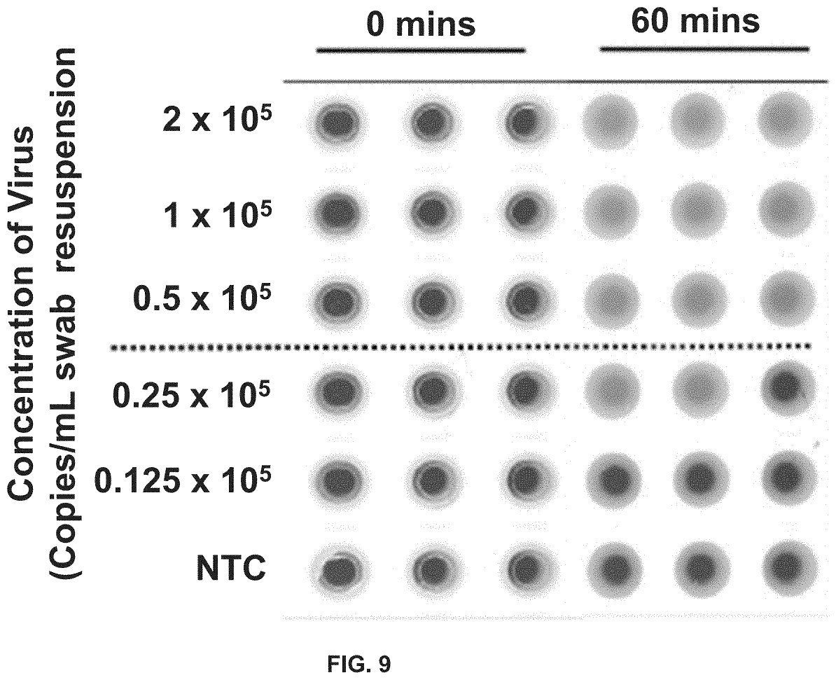

FIG. 9 illustrates the limit of detection in a bovine nasal swab that was re-suspended in about 1 mL water. Heat-inactivated SARS-CoV-2 was spiked into water with the re-suspended background mucus and microbiome to obtain the same number of copies/reaction as in the previous example with saliva. 5 μL of sample was added to 20 μL of RT-LAMP. After incubating at 65° C. for about 1 hour, the color changed. The LOD for the primer was about 250 copies/reaction in a volume of 25 μL, which is equivalent to about 5 k copies/mL of nasal swab resuspension.

### Example 9—Limit of Detection on Paper

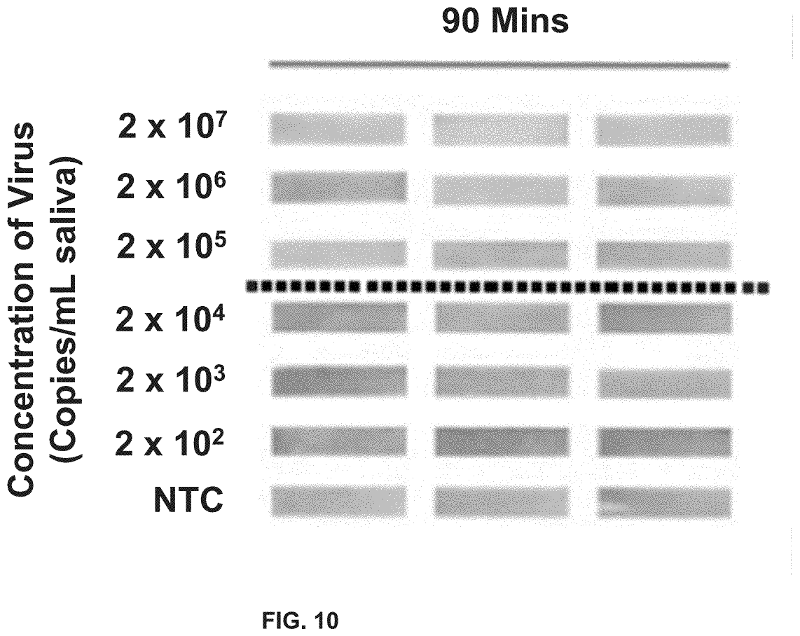

FIG. 10 illustrates the limit of detection on paper. 20 μL of RT-LAMP reagents were added to Grade 1 chromatography paper. Heat-inactivated SARS-CoV-2 was spiked into the 100% pooled saliva with serial dilutions of the virus. 15 μL of about 100% saliva was added to each piece of paper. After incubating at 65° C. for 90 minutes the color changed. The LOD for the primer was about 3 k copies/reaction in a volume of 15 μL saliva, which is equivalent to about 20 k copies/mL of saliva.

Reagent Compositions that Facilitate LAMP Analysis on Paper

### Example 10—Sample Reagents

In one example, the reagents were included as shown in Table A1. In another example, the reagents were included as shown in Table A2.

TABLE A-1
Reagents

Deoxynucleotide (dNTP) Solution Set (100 mM of
each individually dATP, dCTP, dGTP, and dTTP)
Deoxynucleotide (dNTP) Solution Mix
TWEEN ® 20
Bst 2.0 WarmStart ® DNA Polymerase
Bst 3.0 DNA Polymerase
WarmStart ® RTx Reverse Transcriptase
Tris-HCl
Primer sets
Target DNA for QA testing

TABLE A-2
Reagents

Potassium chloride (
Magnesium sulfate
Deoxynucleotide triphosphate (dNTP)
Bst 2.0 DNA Polymerase
WarmStart ® RTx Reverse Transcriptase
Phenol Red
Antarctic Thermolabile uracil DNA glycosylase
(UDG)
Polysorbate 20
Betaine
Bovine Serum Albumin
Trehalose
Nuclease-Free Water
RNase AWAY

### Example 11—Buffer Selection and Concentration

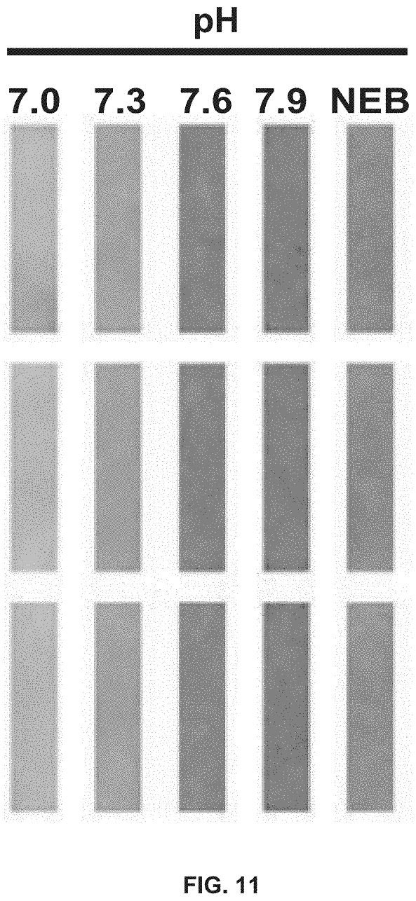

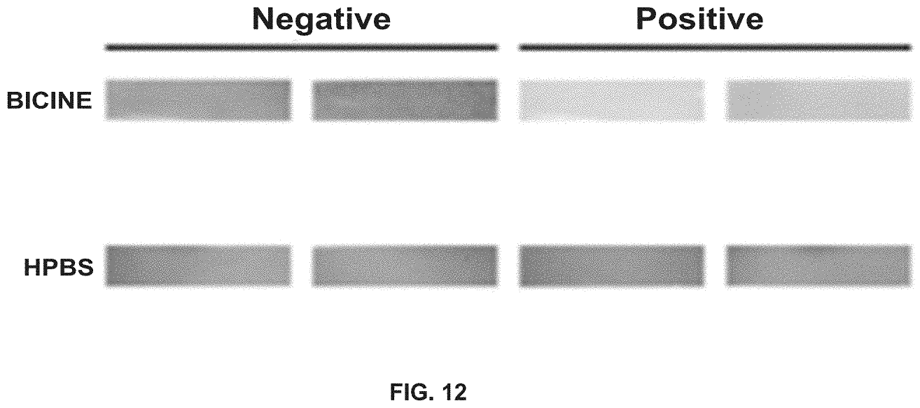

Since the pH of saliva can vary from sample to sample, a buffer was used on the paper-based device to maintain a consistent starting pH. For phenol red, a pH of 7.6 was a suitable starting point to enhance the colorimetric transition as illustrated in FIG. 11. A few buffers having a pKa of about 8 were screened because the starting pH of 7.6 was close to the limits of the buffering range to allow a color change when amplification happens. 10 mM of BICINE buffer was used for the paper-based assay as illustrated in FIG. 12.

### Example 12—Effect of Primers on Reaction Speed

In order to increase the speed of the RT-LAMP reaction, the inclusion of multiple primer sets in the fluorescent RT-LAMP reaction mix was investigated. The investigation was carried out in water using NEB LAMP fluorescent dye as a fluorometric indicator. The inclusion of multiple primer sets did not seem to increase the reaction speed significantly. Rather, the reaction proceeded primarily at the speed of the primer set that had the fastest reaction time when used in isolation.

### Example 13—Sample LAMP Protocol, Reagents, Validation, and Troubleshooting

### Sample Lamp Protocol 13-A:

### Primer Mix

1. Obtain all 6 diluted primers from the freezer; 2. Mix 80 μl of FIP, 80 μl of BIP, 20 μl of FB, 20 μl LB, 10 μl of F3 and 10 μl of B3 in a tube; 3. Add enough PCR-grade water to reach 500 μl.

### LAMP

1. Obtain the NEB Bst 2.0 Warmstart kit and the primer mix; 2. While the reagents thaw and after at least 5 minutes of spraying the DNAway, wipe the surfaces with a Kimwipe; 3.

Label all the PCR tubes needed with the DNA sample and primers that will be used. Make sure to add a negative control which will not have DNA added; 4. Add 5 μl of PCR-grade water (or dye), 12.5 μl of NEB Bst 2.0 Warmstart kit and 2.5 μl of primer mix per reaction. A master mix can be made for however many reactions will be run; 5. If 5 μl of EBT dye are added, it should be in 1500 μM concentration so that the final concentration ends up being 300 μM; 6. The reactions with no DNA should have an extra 5 μl of PCR-grade water added and not opened again until they have to be loaded on a gel; 7. Once ready, the PCR tubes should be put in the PCR tray previously left in the pass-through chamber and carried out to BRK 2037; 8. Once in BRK 2037, obtain the sample DNA from the −20° C. freezer; 9. Spray your hands with DNAway spray and rub your hands around the DNA sample tube so that it is covered in the spray as well; 10. Add 5 μl of the DNA sample where appropriate and close the tubes. Never open 2 DNA tubes at the same time and close the PCR tubes right after adding the DNA; 11. Put the samples in a thermocycler set at 65° C. for 1 hour and 80° C. for 5 minutes (samples may be kept in at −20° C. overnight after this operation).

### Sample Reagent Concentrations 13-B:

Colorimetric RT-LAMP master mix can be: KCl (50 mM), MgSO4 (8 mM), dNTP mixture (1.4 mM each dNTP), Bst 2.0 WarmStart® DNA Polymerase (0.32 U/μL), WarmStart® RTx Reverse Transcriptase (0.3 U/μL), Phenol red (0.25 mM), dUTP (0.14 mM), Antarctic Thermolabile UDG (0.0004 U/μL), Tween® 20 (1% v/v), betaine (20 mM), BSA (500 μg/mL), and trehalose (10% w/v).

These components were titrated each from 0.25× liquid concentration to 5× or higher for the paper LAMP assay. The concentration was determined by the speed of the LAMP reaction, the contrast between positive and negative LAMP results at 60 minutes reaction time, and the reduced amount of non-specific amplification.

To determine the concentration of protein stabilizing additives, D-(+)-trehalose dihydrate was titrated from 0% to 15% w/v using increments of 5% and lyophilized BSA was titrated from 0 to 1.25 mg/mL using increments of 0.2 mg/mL. The concentrations for trehalose and BSA were 10% w/v and 0.626 mg/mL, respectively.

### Sample Lamp Protocol 13-C:

### Reagents

Reagents as shown in Table A-2 of Example 10.

### Equipment

Forceps, 0.5-10 μL Pipette, 2-20 μL Pipette, 20-200 μL Pipette, 100-1000 μL Pipette, Ahlstrom-Munksjö Grade 222, pH probe, a heat source that could reach 65° C. (e.g., incubator, water bath), PCR hood.

### Sanitization:

Spray pipettes and all workbench (PCR hood) surfaces with RNase AWAY. Wipe thoroughly after applying RNase AWAY. The RNase AWAY can interfere with the reaction if there is any remaining. Use separate rooms for manufacturing the paper-based device and loading the samples to aid in the prevention of cross-contamination. Pre-cut 5 mm×6 mm chromatography paper.

### LAMP Preparation:

1. Prepare 2×LAMP mix as indicated in Table 13B-1 inside a PCR hood. 2. Adjust pH with 1M KOH (approximately 1-2 μL) to pH ˜7.5-8.0 (a red but not pink color). Does not need to be precise. After adjusting pH, 2×LAMP Mix can be stored at −20° C.

TABLE 13B-1
2X LAMP Mix

| | | | Stock | | Final | |
|---|---|---|---|---|---|---|
| | | | concen- | | concen- | |
| Components | Volume | Unit | tration | Unit | tration | Unit |
| KCL | 100 | μL | 1000 | mM | 100 | mM |
| MgSO₄ | 160 | μL | 100 | mM | 16 | mM |
| dNTPs | 280 | μL | 10 | mM | 2.8 | mM |
| Bst 2.0 DNA | 10.8 | μL | 120 | U/μL | 1.296 | U/μL |
| Polymerase | | | | | | |
| RTx Reverse | 40 | μL | 15 | U/μL | 0.6 | U/μL |
| Transcriptase | | | | | | |
| Phenol red | 20 | μL | 25 | mM | 0.2 | mM |
| dUTPs | 2.8 | μL | 100 | mM | 0.28 | mM |
| Antarctic | 0.4 | μL | 1 | U/μL | 0.0004 | U/μL |
| Thermolabile | | | | | | |
| UDG | | | | | | |
| Polysorbate 20 | 100 | μL | 20 | % | 2 | % |
| Nuclease-Free | 286 | μL | — | — | — | — |
| Water | | | | | | |
| Total | | | | | 1000 | μL |

3. Prepare a master mix according to Table 13B-2.

TABLE 13B-2
Complete mix for paper LAMP

| | | | Stock | | Final | |
|---|---|---|---|---|---|---|
| | | | concen- | | concen- | |
| Components | Volume | Unit | tration | Unit | tration | Unit |
| 2× LAMP Mix | 125 | μL | — | — | — | — |
| 10× Primer Mix ^{a} | 25 | μL | — | — | — | — |
| Betaine | 1 | μL | 5 | M | 20 | mM |
| BSA | 3.13 | μL | 40 | mg/mL | 0.626 | mg/mL |
| Trehalose | 36 | μL | 689 | mg/mL | — | — |
| Water | 9.2 | μL | | | | |
| Total | | | | | 200 | μL |

^{a} Stocks of the LAMP primers can be made at a workable concentration in water for ease of setup. A 10× Primer Mix containing all 6 LAMP primers. 10× Primer mix: 16 μM FIP/BIP, 2 μM F3/B3, 4 μM Loop F/B can be made.

4. Adjust pH to 8.0 with 0.1M KOH. Use a micro-pH electrode. 5. Mix thoroughly.

Lay paper pads out on a clean surface inside the PCR hood. Add 30 μL of complete mix on pre-cut grade 222 paper pads. 6. Dry under PCR hood at room temperature for 60 minutes. 7. After drying, collect paper pads into a clean centrifuge tube or a clean re-sealable plastic bag.

### Sample Loading

1. Spray down the working bench with RNase AWAY and clean it with wipers. 2. Take out templates (DNA, RNA, heat-inactivated virus) from the freezer. 3. Lay out the reaction pads on a clean surface. You can lay your pads on a new transparency film and discard it after use. 4. Prepare negative control pads first. Reconstitute pads with 25 μL non-template solvent (water, saliva). The reconstitution process should be gentle, avoid washing out the regents from the pad. 5. Place the negative control pads inside a clean container (e.g., 1″×1″ resealable plastic bag, centrifuge tube) using forceps. 6. Dilute template with solvent into desire concentration. 7. Lay out more reaction pads and reconstitute pads with 25 μL diluted template. 8. Place the positive pads inside a clean container (e.g., 1″×1″ resealable plastic bag, centrifuge tube) using forceps. 9. Clean up the workspace and bring the pads for imaging and incubating.

### Imaging and Incubation:

Note: There are multiple imaging methods (e.g., time-lapse video, scanning) and heat sources (e.g., incubator, water bath). In this protocol, a tabletop scanner and a microbiological incubator can be used. 1. Arrange the pads on top of the scanner. Scan the pads before the reaction (0 min). 2. Preheat the incubator to 65° C. 3. Place the pads into the incubator. Separate the pads. The heating uniformity can affect the result consistency. 4. Take out pads and repeat scanning at different time points (usually every 30 minutes). 5. After final scanning, discard the reaction pads inside biohazard trash.

### Validation:

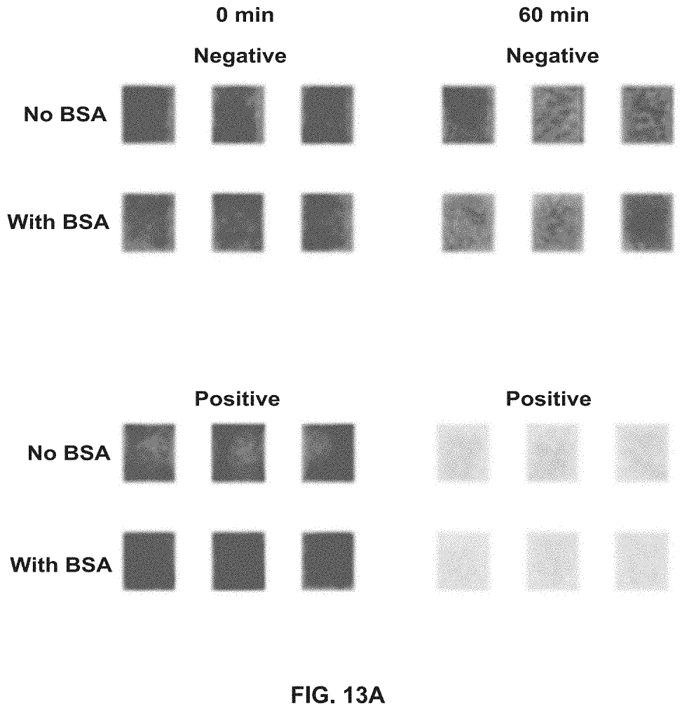

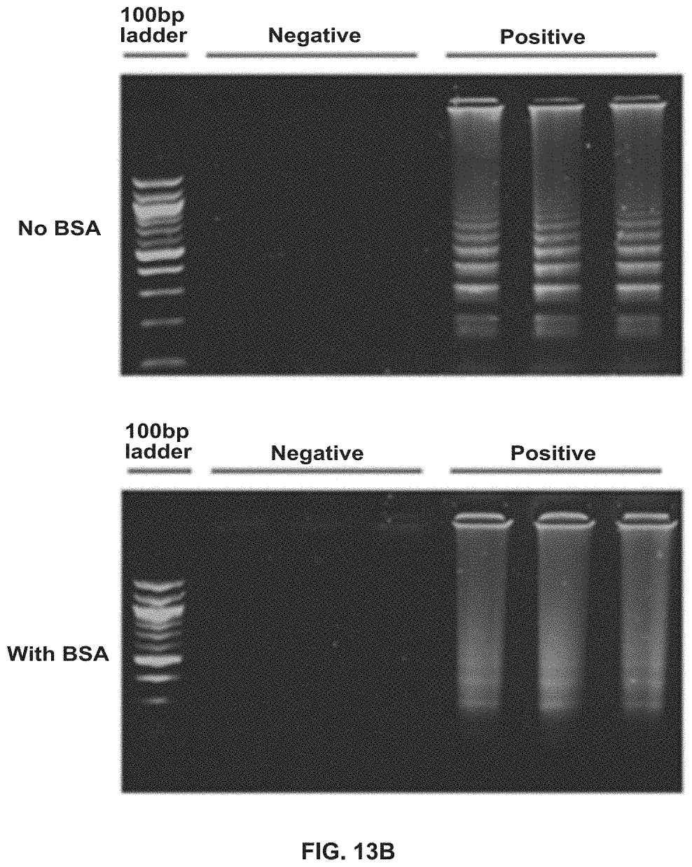

To verify the occurrence of LAMP amplification, each reaction pad was transferred to a clean 1.5 mL microcentrifuge tube. 100 μL Buffer EB was added to each tube. Reaction pads were submerged in Buffer EB overnight for eluting nucleic acids. Gel electrophoresis (2% agarose gel) was done with the eluent to verify the occurrence of LAMP amplification. A ladder-like pattern (typical LAMP product pattern) was shown in each positive pad lane while there was no obvious band in each negative lane (FIGS. 13A and 13B).

As illustrated in FIGS. 13A and 13B, paper LAMP validation was performed. As shown in FIG. 13A, LAMP on paper with two conditions (with and without BSA in the reaction mix) was performed. As shown in FIG. 13B associated gel electrophoresis (2% agarose) was performed. The orf7ab.1 primer set targeting SARS-CoV-2 was used. Negative reaction pads were reconstituted with 25 μL nuclease free water. Positive reaction pads were reconstituted with 25 μL 400 copies/μL heat-inactivated SARS-CoV-2 virus. Heating was carried out in an incubator set at 65° C. and scanned in a flatbed scanner.

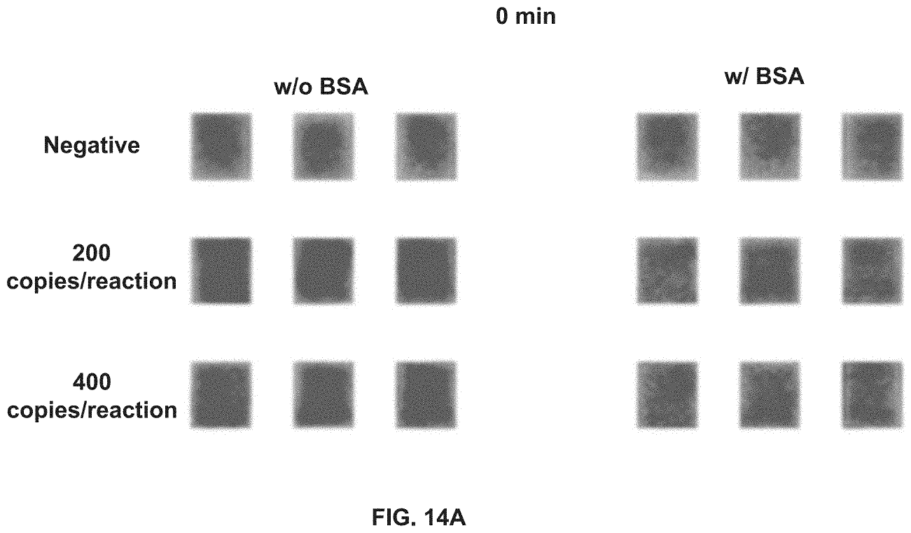

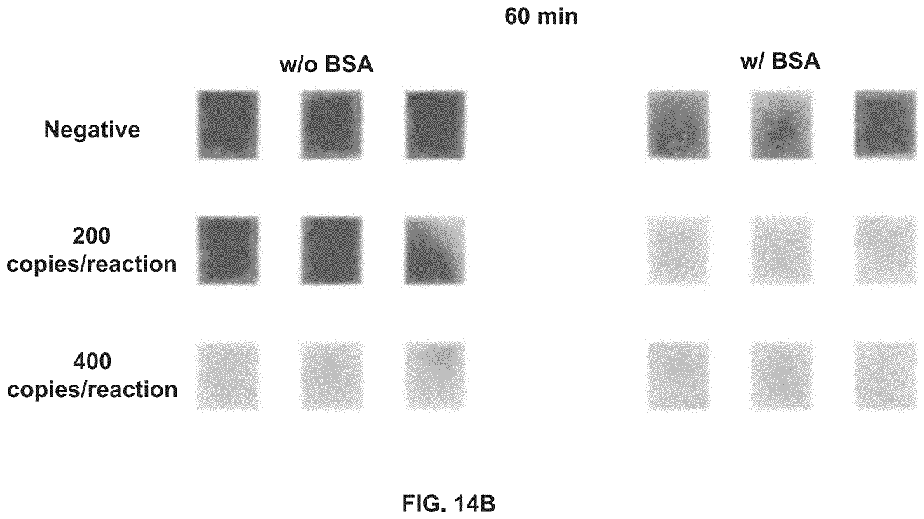

BSA is a reagent that can be used in the LAMP mix. Adding BSA can speed up reactions and increase sensitivity, as illustrated in FIG. 14A and FIG. 14B. In these reactions, a low template concentration LAMP on paper was performed subject to two conditions (with and without BSA in the reaction mix). FIG. 14A shows a 0-minute time point. FIG. B shows a 60-minute time point. The orf7ab.1 primer was used in this experiment. Negative reaction pads were reconstituted with 25 μL nuclease free water. Positive reaction pads were reconstituted with 25 μL 8 copies/μL and 16 copies/μL heat-inactivated SARS-CoV-2 virus (to reach a final concentration of 200 copies/reaction and 400 copies/reaction), respectively.

However, BSA can also introduce pH variations to the device. FIG. 13A and FIG. 14B show that after incubation (60 min) negative paper pads containing BSA have a yellowish edge. After elution and running the eluent with gel electrophoresis, there was no DNA product visible on the gel, as shown in FIG. 13B, indicating that the yellow color at the edge is not caused by off-target amplification or contamination. A heterogeneous distribution of BSA can lead to the yellow color at the edges upon application of heat.

### Troubleshooting:

Unusual pink color on paper pads: During the process of preparing LAMP paper pads, there can be unusual pink spots different from the surrounding color, which can be caused by residual RNase AWAY either directly sprayed onto the pads and/or transferred via the forceps. RNase AWAY can degrade any RNA/DNA template added. If this occurs, thoroughly dry all equipment and surfaces, cut new 5×6 mm paper pads, and restart the ‘LAMP preparation’ section from Operation 5.

Overflowing of reagents after pad reconstitution: During the sample loading operation, the pad can be unable to absorb the entire sample volume added to it for reconstitution. The template concentration may not be accurately represented by overflowed pads. Overflowing can be caused by insufficient drying of the pads. If this occurs: 1) dry for a longer time, 2) use an enhanced drying method such as heat drying (place on a clean microbiological incubator at 37° C.; do NOT set the temperature higher than 45° C. to prevent activation of the Bst 2.0 WarmStart® polymerase) or convective drying (use small fans to enhance airflow during drying), or 3) reduce reconstitution volume to 20 μL.

Negative controls exhibit color change: During the imaging and incubation operation, the negative pads can change at the same time or shortly after the sample-containing pads. This can be caused by either primer dimerization/non-specific amplification or carryover contaminants of previous LAMP reactions. To resolve, validate the primer in liquid-based LAMP prior to using them on paper. To control carryover contaminations, 1) implement dUTPs and UDG in all LAMP reactions, 2) maintain separate working stations for LAMP mixture preparation and sample addition, and 3) aliquot reagent stocks and use new aliquots if contamination is suspected to have occurred. Over-incubating the reaction can also induce non-specific amplification. Do not exceed an incubation time of 75 minutes.

Sample pH and buffering capacity will influence colorimetric readouts: Since phenol red is a pH indicator, the pH of the sample and its buffering capacity can have a significant impact on the assay. It has been confirmed that saliva concentrations of 5-10% v/v (diluted with water) work with the reagent composition presented here. 5% saliva was selected because that concentration has a faster response time and produces consistent results. Human saliva has a sophisticated buffering system that includes bicarbonate, phosphate, and proteins, which prevents pH change (and thus color change) at high saliva concentration. The paper LAMP device with nasal swab resuspended in water was tested and not show any inhibition from the sample matrix. Colorimetric readouts can also be hampered by buffered salt solution (e.g., transport media).

### Example 14—Limit of Detection on Paper of Untreated Saliva with Inactivated Virus

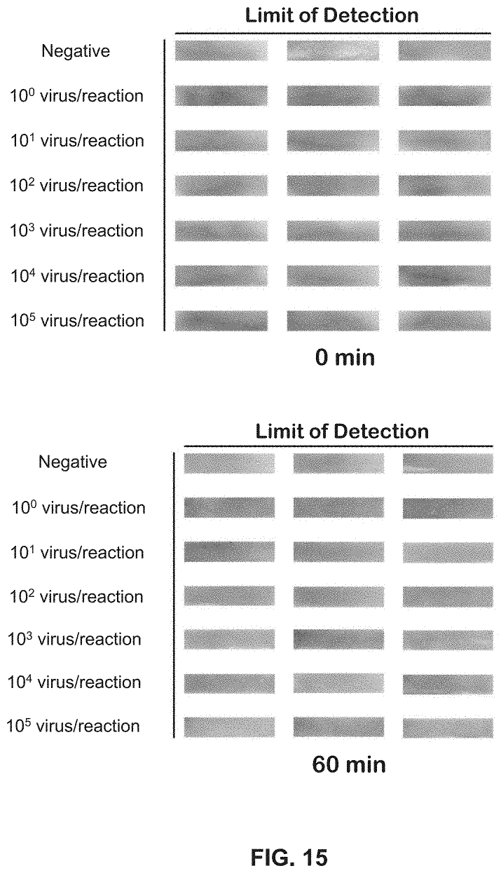

As illustrated in FIG. 15, whole untreated saliva with heat inactivated SARS-CoV-2 virus validated the limit of detection of about 20 copies per μL saliva. Various sample concentrations including 20 copies/μL (1× LoD—10 samples), 40 copies/μL (2× LoD—10 samples), 100 copies/μL (2 samples), 1000 copies/μL (2 samples), 10,000 copies/μL (2 samples), 100,000 copies/μL (2 samples), 1,000,000 copies/μL (2 samples) were created using aliquots of pooled saliva (30 aliquots) as the negative samples. The results were confirmed using image processing.

Reagent Compositions that Maximize PH-Sensitive Signal Output

### Example 15—Sample LAMP Dyes

TABLE B
Dye

Phenol red solution
Litmus
Bromothymol Blue
Nitrazine Yellow
Cresol Red, Sodium salt
Curcumin
Brilliant Yellow
m-Cresol Purple
α-Naphtholphthalein
Neutral Red
Acid fuchsin
Acid fuchsin, Calcium salt

### Example 16

Fluorescent reporters would use an additional ultraviolet (UV) light source to be read without specialized instrumentation. However, a colorimetric assay using phenol red as an indicator would not use UV light, and can be interpreted by the naked eye. Polymerization of DNA produces protons and phenol red is responsive to pH. Diluted saliva (5% final concentration) was used to overcome the buffering capacity of saliva to measure changes in pH. Diluting saliva to a 5% final concentration also reduced the concentration of interferents (e.g., RNase).

Incorporating carrier DNA and guanidine hydrochloride also enhanced the LoD and provided a colorimetric response that was comparable in water and in saliva. The mechanism by which carrier DNA increase the LAMP results was unclear. This mechanism was explored by using NEB 1 kb DNA ladder (NEB-N3232L). Different concentrations of carrier DNA were used (0.3 ng/μl and 0.75 ng/μl) to study the effect on the LoD. Guanidine chloride (40 mM) was also used. The pH of the complete master mix was maintained at 7.6., and the same condition was also tested without carrier DNA. Fresh saliva (5%) with a pH of 6.5 was used to test the effect of carrier DNA along with guanidine chloride with the heat-inactivated SARS-CoV-2 at a concentration range of from 1000 copies to 62.5 copies/reaction (FIG. 6D).

Guanidine hydrochloride was reported to increase the sensitivity of LAMP. Its performance with our primer set was tested by adding 40 mM of guanidine hydrochloride to the NEB Warmstart™ colorimetric master mix with a pH of 7.6. Pooled saliva (5%) with a pH of 6.5 was used to test the effect of guanidine chloride with the heat-inactivated SARS-CoV-2 with a concentration range of from 1000 copies to 62.5 copies/reaction. This same composition was also tested without adding guanidine chloride as a control. Guanidine chloride increased the replicate sensitivity and has a consistent amplification across the replicate (FIG. 6E).

Due to the different mechanisms in which phenol red and fluorescent dye report LAMP-based nucleic acid amplification, these differences in signal measurement over time were investigated. Reactions were prepped on FrameStar 96-well skirted optical bottom plates with a combination of Warmstart™ Colorimetric LAMP 2× Master Mix and LAMP Fluorescent Dye, sealed with Thermo Scientific™ Adhesive Plate Seals, and placed in a Clariostar™ Plus Microplate Reader (BMG) to for incubation and measurement of absorbance and fluorescent intensity. Color change over time was expressed as a ratio of A_{432 nm}/A_{560 nm}. Absorbance values were baseline-corrected by subtracting A_{432 nm} and A_{560 nm} by A_{620 nm}.

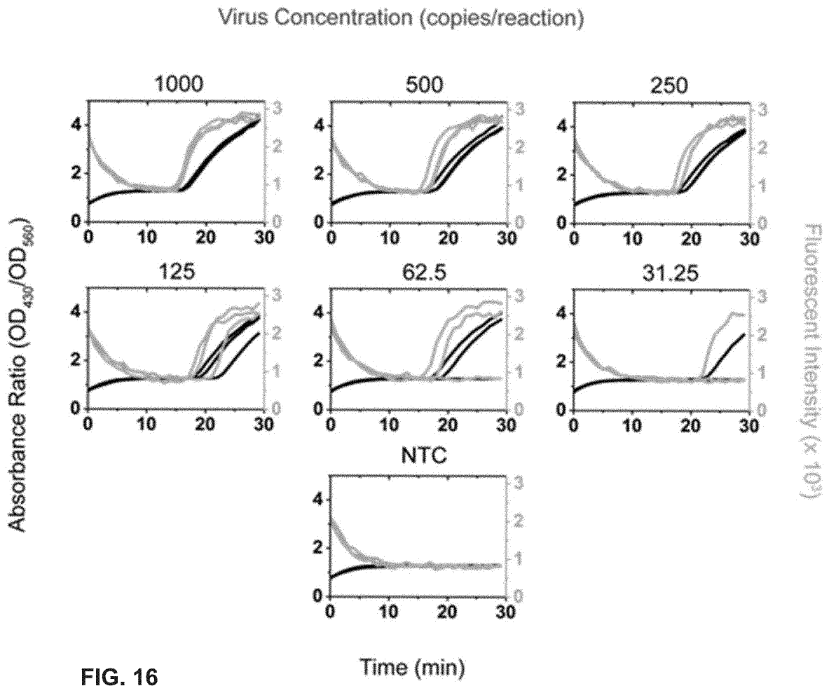

Based on FIG. 16, color changes in reactions occurred later compared to fluorescence changes. Colorimetric and fluorometric data was collected on BMG CLARIOstar® Plus plate reader. Reaction base mix consisted of NEB 2× Colorimetric LAMP master mix, 2.5 μL of primer mix, and 5 μL of a 1:100 dilution of NEB LAMP Fluorescent Dye (NEB B1700A). The chamber temperature of the plate reader was allowed to equilibrate to 65° C. prior to inserting the plate. 5 μL of heat-inactivated virus diluted in water was added to the reaction base mix to result in the final reaction concentrations as indicated (positive reactions). For NTC reactions, 5 μL of nuclease-free water was added to the reaction base mix. This difference in change suggested that pH-based reporters give a slower response to LAMP-based DNA amplification than fluorescent reporters.

### Example 17

Paper can be scaled up to millions of devices, but when RT-LAMP reagents were placed on paper, the paper changed color even when a negative control was used even though no amplification was occurring. One possibility for this color change is oxidation of cellulose caused by the heat and the oxidizing nature of ammonium sulfate present in the RT-LAMP mixture. Another possibility for this color change is acidification of the reagents due to degassing of ammonia from the RT-LAMP mixture. Eliminating ammonium sulfate maintained the color for a negative control. Increasing the concentration of phenol red, which acts as an antioxidant, also maintained the color for the negative control as illustrated in FIG. 11.

### Example 18—Screening of Colorimetric Dyes

Three classes of colorimetric indicators were evaluated for a paper-based assay: (i) magnesium colorimetric indicators, (ii) pH colorimetric indicators, and (iii) DNA intercalating colorimetric indicators.

For magnesium indicators, Calmagite (CAS #3147-14-6), Xylidly blue I (CAS #14936-97-1), Chlorophosphonazo III (CAS #1914-99-4), o-Cresolpthalein Complexone (CAS #2411-89-4), Eriochrome® Black T (EBT, CAS #1787-61-7), and Hydroxynapthol blue (HNB, CAS #63451-35-4) were screened.

For pH indicators, Bromothymol Blue (CAS #76-59-5), Acid Fuchsin (CAS #3244-88-0), Nitrazine yellow (CAS #5423-07-4), Cresol red (CAS #1733-2-6), Cresol red sodium salt (CAS #62625-29-0), Curcumin (CAS #458-37-7), Phenol red (CAS #143-74-8), Phenol red sodium salt (CAS #34487-61-1), Brilliant yellow (CAS #3051-11-4), o-Cresolphthalein (CAS #596-27-0), m-Cresol purple (CAS #2303-01-7), m-Cresol purple sodium salt (CAS #62625-31-4), α-Naptholphthalein (CAS #596-01-0), and Neutral red (CAS #553-24-2) were screened.

For DNA intercalating dyes, Crystal violet (CAS #548-62-9) was screened.

Many magnesium indicators did not produce a consistent color change on paper. The metal ion indicators (calmagite and EBT) interacted with magnesium(II) ions in solution whose concentration decreased throughout the RT-LAMP experiment due to the formation of magnesium pyrophosphate, a byproduct of the polymerase reaction.

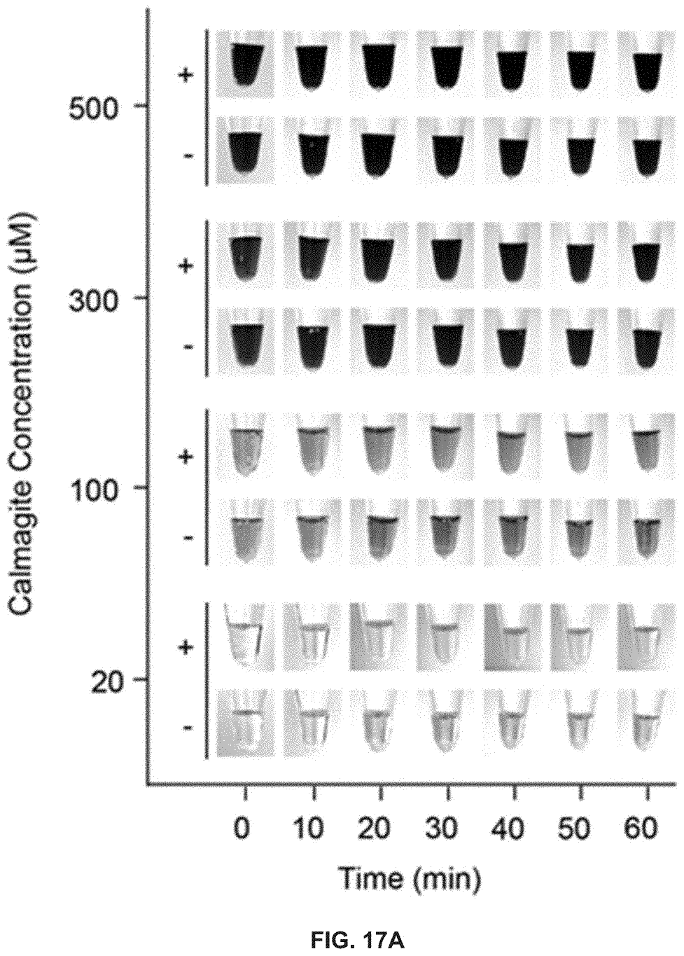

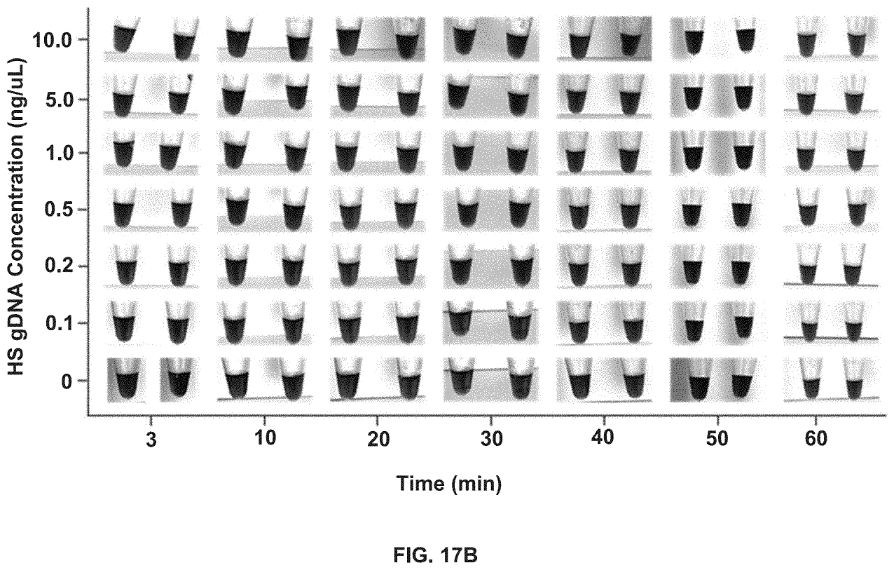

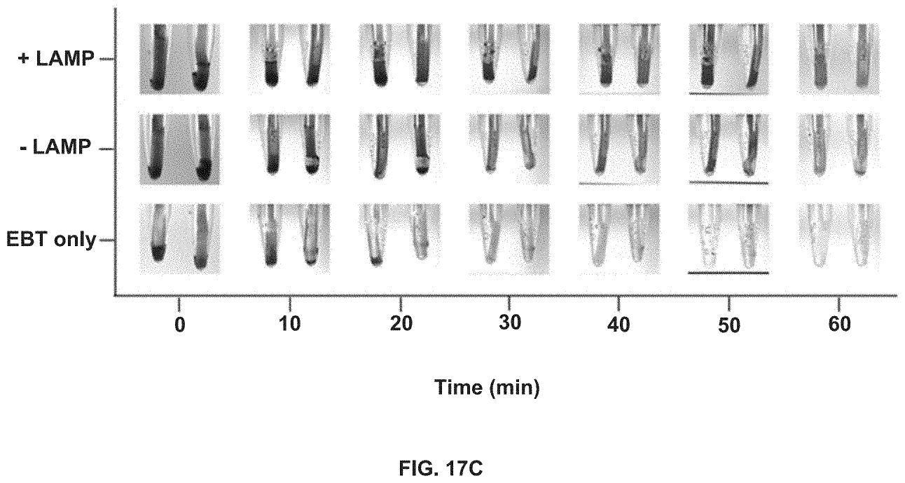

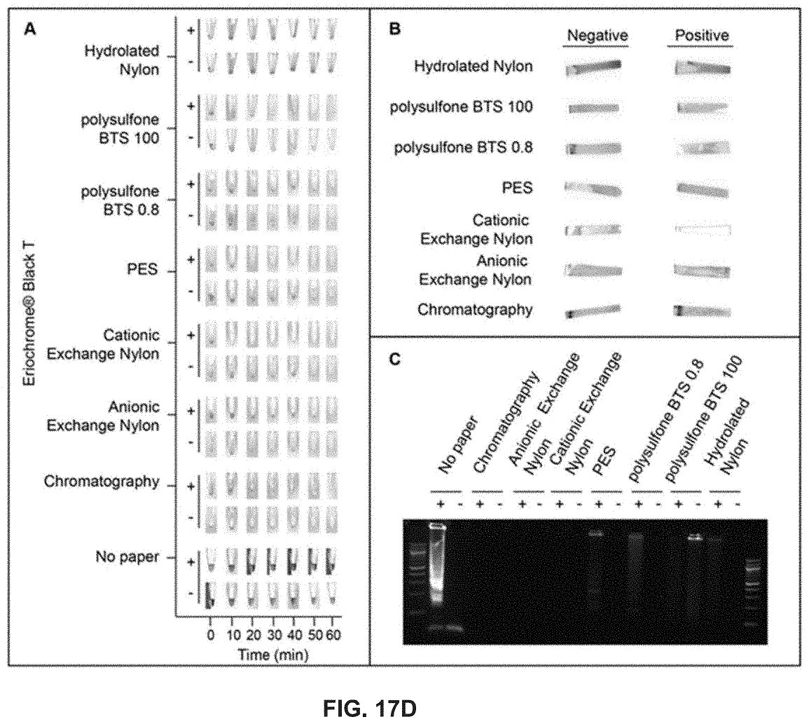

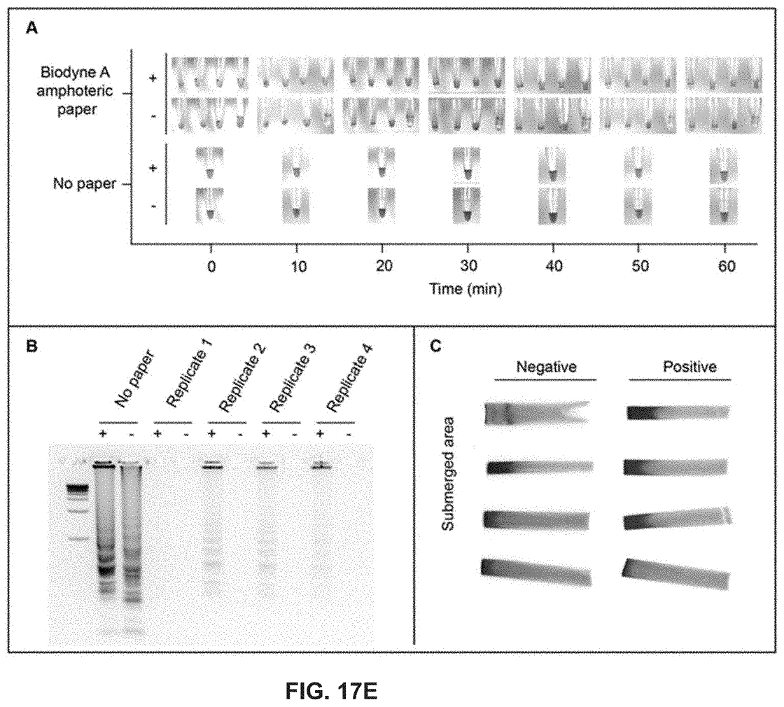

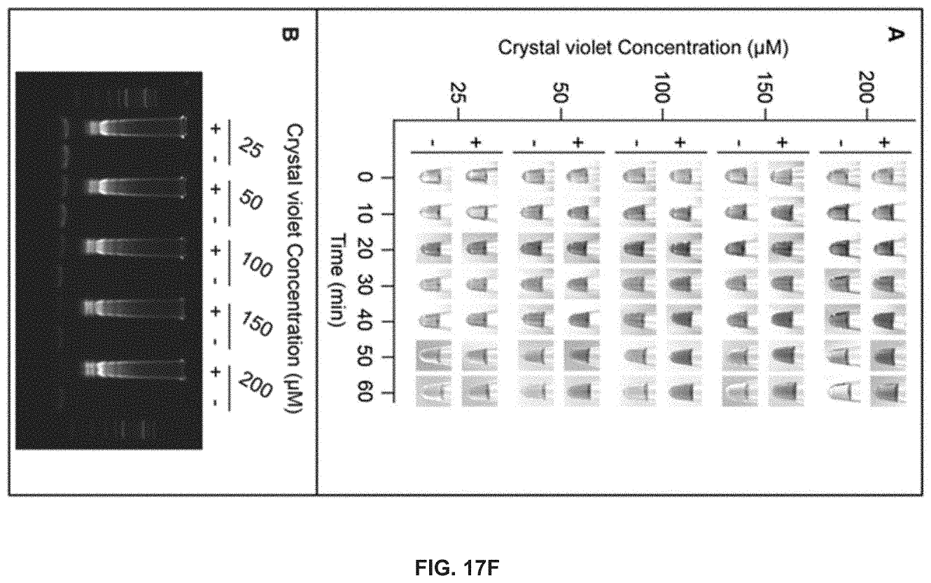

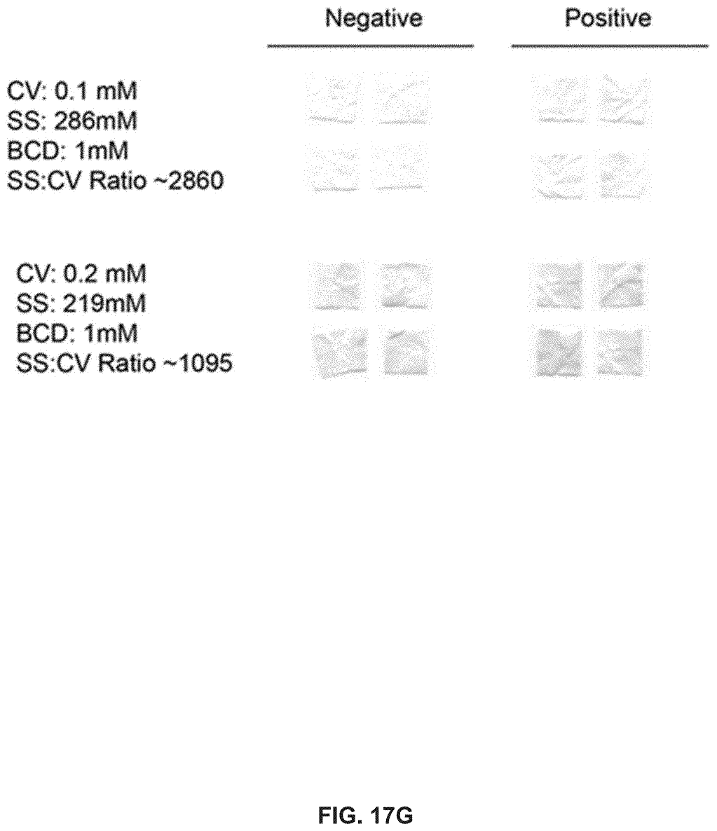

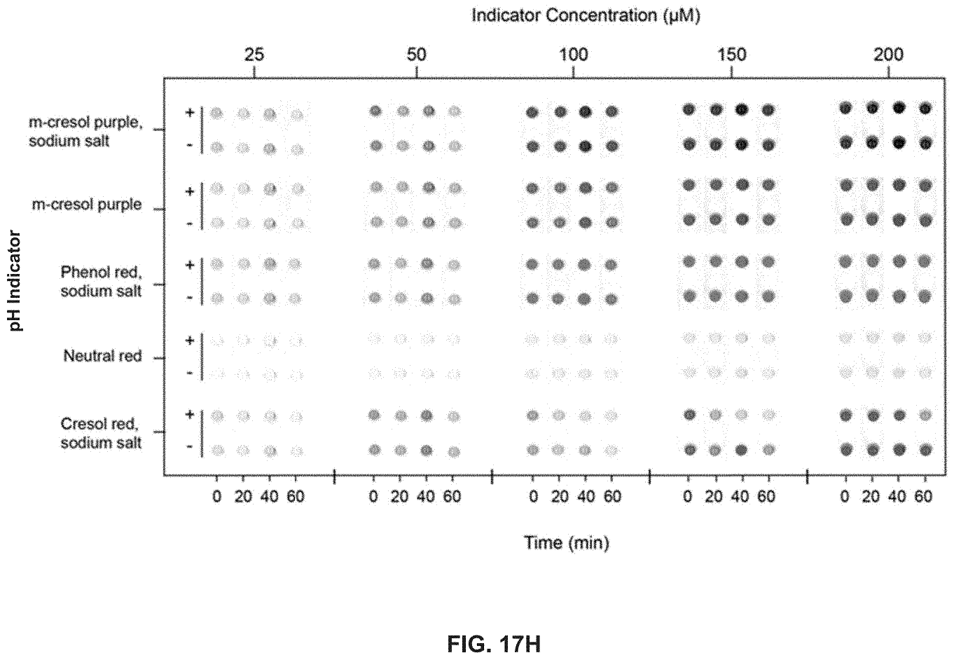

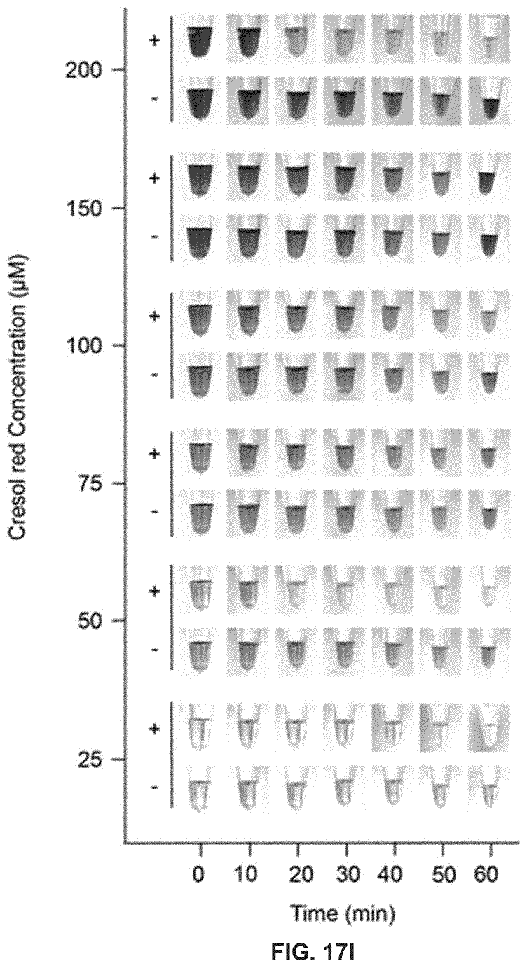

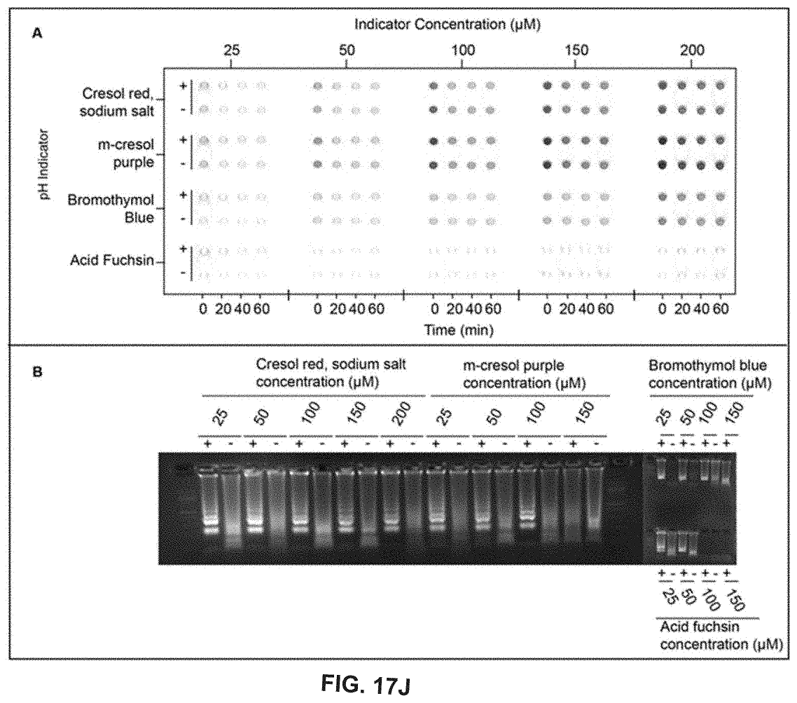

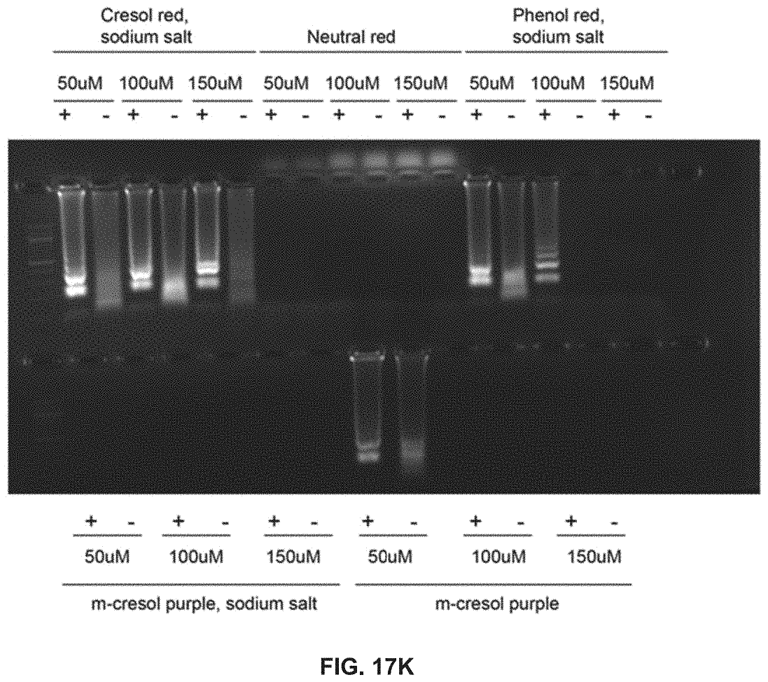

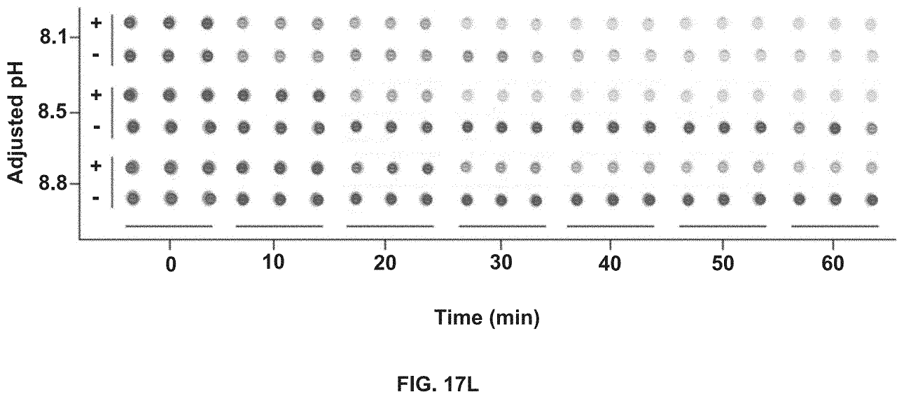

FIG. 17A shows the colorimetric response of calmagite at varying concentrations throughout the LAMP reaction using genomic DNA as template. LAMP detection was performed with increasing concentrations of calmagite (magnesium indicator). The lolB.3 primer set targeting Histophilus somni genomic DNA was used. Positive reactions were spiked with 5 μL of HS gDNA at a concentration of 0.2 ng/μL. Negative reactions used 5 μL of nuclease-free water. The total reaction volume was 25 μL. Reactions were prepared using 12.5 μL of NEB Warmstart 2× master mix, 2.5 μL or primer mix, and 5 μL of either template (positive reactions) or water (negative) as above, and 5 μL of Calmagite prepared in water to produce a final concentration as indicated.

At the concentrations tested, a visual change was not detected during the LAMP reaction. EBT showed a detectible color change from violet to a dark blue between the 0 minute and 60 minute time points of the LAMP reaction; however, the color change was not clear, which can interfere with interpretations in clinical settings.

As illustrated in FIG. 17B), LAMP detection was performed with increasing concentrations of Eriochrome® Black T (magnesium indicator). The lolB.3 primer set targeting Histophilus somni genomic DNA (gDNA) was used. Positive reactions were spiked with 5 μL of HS gDNA at a concentration of 0.2 ng/μL. Negative reactions used 5 μL of nuclease-free water. Total reaction volume is 25 μL. Reactions were prepared using 12.5 μL of NEB Warmstart 2× master mix, 2.5 μL or primer mix, and 5 μL of either template (positive reactions) or water (negative) as above, and 5 μL of EBT prepared in water to produce a final concentration as indicated.

Additionally, using LAMP on paper using EBT did not result in a detectable color change. As illustrated in FIG. 17C, LAMP detection was performed with increasing concentrations of Eriochrome® Black T on chromatography paper in PCR tubes. The lolB.3 primer set targeting Histophilus somni genomic DNA was used. Positive reactions were spiked with 5 μL of H. somni gDNA at a concentration of 0.2 ng/μL. Negative reactions used 5 μL of nuclease-free water. Total reaction volume was 25 μL. Reactions were prepared using 12.5 of NEB Warmstart 2× master mix, 2.5 μL or primer mix, and 5 μL of either template (positive reactions) or water (negative) as above, and 5 μL of EBT (300 μM) prepared in nuclease-free water. For EBT reactions, the reaction consisted of 25 μL of EBT (300 μM) prepared in nuclease-free water.

By screening several different types of paper and confirming via gel electrophoresis that amplification was occurring on PES and polysulfone BTS 0.8, a colorimetric change on any paper could not be detected (FIG. 17D and FIG. 17E).

As shown in FIG. 17D, LAMP detection was performed on multiple papers: chromatography grade 1, anionic exchange nylon, cationic exchange nylon, polyether sulfone membrane, asymmetric sub-micron polysulfone (BTS 0.8), asymmetric sub-micron polysulfone (BTS 100) and hydroxylated nylon 1.2. b) Endpoint scans of the papers in panel a at 60 minutes and c) gel electrophoresis (2% agarose) scan of the extracted LAMP products at 60 minutes. The lolB.3 primer set targeting Histophilus somni (HS) genomic DNA was used. Positive reactions were spiked with 5 μL of H. somni gDNA at a concentration of 0.2 ng/μL. Negative reactions used 5 μL of nuclease-free water. Total reaction volume is 25 μL. Reactions were prepared using 12.5 μL of NEB Warmstart 2× master mix, 2.5 μL or primer mix, and 5 μL of either template (positive reactions) or water (negative) as above, and 5 μL of EBT (300 μM) prepared in nuclease-free water. Papers (as indicated) were placed in a a PCR tube containing 25 μL of reaction and was wicked by the paper over the course of reaction. After 60 minutes, the papers were removed and scanned. Gel was extracted using 30.

As illustrated in FIG. 17E, results were generated for overtime in a) PCR tubes, b) gel electrophoresis (2% agarose) of the extracted DNA from papers at 60 minutes, and c) scanned papers at 60 minutes. The lolB.3 primer set targeting Histophilus somni (HS) genomic DNA was used. Positive reactions were spiked with 5 μL of H. somni gDNA at a concentration of 0.2 ng/μL. Negative reactions used 5 μL of nuclease-free water. Total reaction volume is 25 μL. Reactions were prepared using 12.5 μL of NEB Warmstart 2× master mix, 2.5 μL or primer mix, and 5 μL of either template (positive reactions) or water (negative) as above, and 5 μL of EBT (300 μM) prepared in nuclease-free water. Biodyne A amphoteric paper were placed in a PCR tube containing 25 μL of reaction and was wicked by the paper over the course of reaction. After 60 minutes, the papers were removed and scanned.

Additionally, it was difficult to stabilize the Crystal violet indicator in solution for the RT-LAMP reaction. For Leuco crystal violet (LCV), an unstable derivative of crystal violet, excess sodium sulfite was used to maintain stability colorlessness in solution. To solubilize in water, LCV used sodium sulfite (SS) and beta-cycLoDextrin (BCD). Upon binding to dsDNA, LCV converted back to crystal violet (e.g., violet in solution). Consequently, throughout the RT-LAMP reaction as more dsDNA was produced as a result of amplification, the solution was expected to change color from colorless to violet. When LAMP reactions were performed at varying concentrations of CV, however, changes in color occurred in both positive and negative reactions.

As shown in FIG. 17F, LAMP detection was performed with intercalating dye, Crystal violet in solution and b) associated gel electrophoresis (2% agarose) scan of the products at 60 minutes. The lolB.3 primer set targeting H. somni gDNA was used. Positive reactions were spiked with 5 μL of H. somni gDNA at a concentration of 0.2 ng/μL. Negative reactions used 5 μL of nuclease-free water. Total reaction volume was 25 μL. Reactions were prepared using 12.5 μL of NEB Warmstart 2× master mix, 2.5 μL or primer mix, and 5 μL of either template (positive reactions) or water (negative) as above, and 5 μL of Crystal violet prepared in nuclease-free water to result in a final concentration as indicated. Papers were placed in a PCR tube containing 25 μL of reaction and was wicked by the paper over the course of reaction. After 60 minutes, the papers were removed and scanned. Sodium sulfite and cyclodextrin were used to solubilize crystal violet.

To confirm that amplification was occurring in the positive reactions but not the negative reactions, the RT-LAMP solution was run in a 2% agarose gel, which showed that the positive reactions showed amplification at all tested concentrations of CV, while the negative reactions did not show amplification at any tested concentrations of CV. Thus, the color change in the negative reactions was caused by the degradation of LCV to CV, not because of binding of amplified DNA.

Further testing for CV on paper at different concentrations of CV, SS, and BCD, provided results that were indistinguishable between negative and positive reactions. As shown in FIG. 17G, endpoint colorimetric scans were performed for LAMP detection with intercalating dye, Crystal violet (CV), on paper. Sodium sulfite (SS) and Beta-cyclodextrin (BCD) were used to solubilize CV. Papers were loaded with 25 μl of LAMP reaction prepared using 12.5 μL of NEB Warmstart™ 2× master mix, 2.5 μL or primer mix, and 5 μL of either template (positive reactions) or water (negative) as above, and 5 μL of CV prepared in nuclease-free water at the indicated concentrations.

Finally, several colorimetric pH indicators which cover a range of about 2 pH units with a color transition pH of about 7.0 were tested to match the anticipated pH range change and transition point of a RT-LAMP reaction. These ranges were chosen based on the initial starting pH of our LAMP colorimetric master mix and also to overcome the buffering capacity of saliva. One exception to this selection process was Acid fuchsin, which covers a range of 3.0 pH and has a color transition pH of 5.0. FIGS. 17H-17K show varying concentrations of the selected pH indicators along with the associated gel electrophoresis results of the LAMP reaction at 60 minutes.

As illustrated in FIG. 17H, RT-LAMP detection was performed with increasing concentrations of cresol red sodium salt, Neutral red, Phenol red sodium salt, m-cresol purple, and m-cresol purple sodium salt in solution (pH indicator). The N.10 primer set targeting the N gene of SARS-CoV-2 was used. Positive reactions were spiked with 5 μL of in-vitro transcribed N gene RNA at a concentration of 0.2 ng/μL. Negative reactions used 5 μL of nuclease-free water. Total reaction volume was 25 μL. Reactions were prepared using 12.5 μL of NEB Warmstart™ 2× master mix, 2.5 μL or primer mix, and 5 μL of either template (positive reactions) or water (negative) as above, and 5 μL of the indicated pH indicator at the designated concentration in nuclease-free water. Reactions were carried out in an incubator and scanned every 20 minutes using a flatbed scanner.

As illustrated in FIG. 17I, LAMP detection with increasing concentrations of Cresol red (pH indicator) was performed. The lolB.3 primer set targeting H. somni gDNA was used. Positive reactions were spiked with 5 μL of H. somni gDNA at a concentration of 0.2 ng/μL. Negative reactions used 5 μL of nuclease-free water. Total reaction volume is 25 μL. Reactions were prepared using 12.5 μL of NEB Warmstart™ 2× master mix, 2.5 μL or primer mix, 5 μL of cresol red resulting in a final reaction concentration as indicated, and 5 μL of either template (positive reactions) or water (negative) as above.

As illustrated in FIG. 17J, RT-LAMP detection was performed with increasing concentrations of Cresol red, sodium salt, m-cresol purple, Bromothymol blue and Acid fuchsin in solution and b) associated gel electrophoresis (2% agarose) scan of products at 60 mins. The N.10 primer set targeting the N gene of SARS-CoV-2 was used. Positive reactions were spiked with 5 μL of in-vitro transcribed N gene RNA at a concentration of 0.2 ng/μL. Negative reactions used 5 μL of nuclease-free water. Reactions were prepared using 12.5 μL of NEB Warmstart 2× master mix, 2.5 μL or primer mix, and 5 μL of either template (positive reactions) or water (negative) as above, and 5 μL of the indicated pH indicator to result in a final reaction concentration as indicated. Heating was carried out in an incubator set at 65° C. and scanned in a flatbed scanner every 20 minutes.

As illustrated in FIG. 17K, endpoint gel electrophoresis scans of the RT-LAMP products (60 mins) were performed on a 2% Agarose gel.

As shown, one pH indicator that produced a distinct colorimetric response between positive and negative reactions was cresol red. Additionally, phenol red (the pH indicator used in NEB's colorimetric RT-LAMP kit) was also evaluated with respect to varying initial pH values resulting from the addition of HCl and KOH to the solution to provide the indicated initial pH value.

As illustrated in FIG. 17L, RT-LAMP detection with Phenol red at pH 8.1, 8.5, and 8.8 in solution (pH indicator) was performed. Adjustments were made with HCl and KOH prior to the addition of template RNA. The N.10 primer set targeting the N gene of SARS-CoV-2 was used. Positive reactions were spiked with 5 μL of in-vitro transcribed N gene RNA at a concentration of 0.2 ng/μL. Negative reactions used 5 μL of nuclease-free water. Reactions consisted of 20 master mix and 5 μL of template as described above (positive) or nuclease-free water (negative). 10 mL of master mix was made with (NH₄)₂SO₄ (20 mM), KCl (100 mM), MgSO₄ (16 mM), dNTP mix (28 mM each dNTP), tween 20 (0.2% v/v). Therefore, phenol red resulted in a higher level of contrast between positive and negative reactions when compared to cresol red.

Consequently, pH indicators with a color change around pH 6.5 had the most consistent and the most contrasting color change (e.g., Phenol red).

### Example 19—Effect of Starting pH on Paper

To evaluate the color stability of incorporating drying into our process, the pH of the LAMP master mix was adjusted to 8.0, 8.5, or remained unadjusted (e.g., 7.6) and the water or synthetic RNA (N gene, 0.2 ng/μL) used for rehydration was also adjusted to 8.0, 8.5, or remained unadjusted (e.g., 5.5).

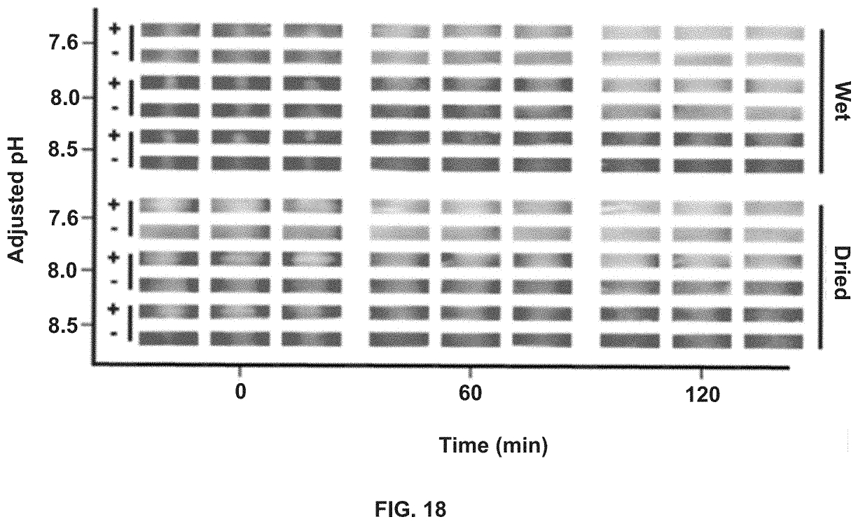

As illustrated in FIG. 18, pH 7.6 is the unadjusted pH of the RT-LAMP reaction mixture. Wet setup indicates 5 μL of synthetic RNA (N gene, 0.2 ng/μL, ‘+’) or water (‘−’) were added immediately after adding 20 μL of LAMP reaction master mix. Dried setup indicates paper strips were left to dry for 30 minutes at room temperature after applying 20 μL LAMP master mix and then rehydrated with 25 μL synthetic RNA (‘+’) or water (‘−’).

Adjusting the pH to 8.0 resulted in a better color stability in the negative controls, whereas a pH of 8.5 was too high to allow for discernible color change the standard and the dry set ups after 120 minutes of incubation. When the pH was left unadjusted, the color changed even when a control was loaded.

### Example 20—Effect of Trehalose and Tween 20 on RT-LAMP Colorimetric Response

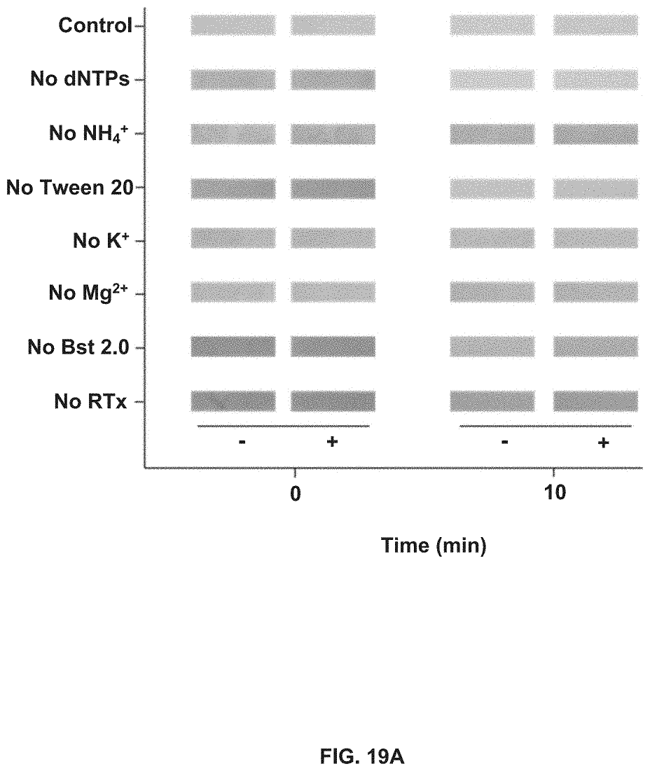

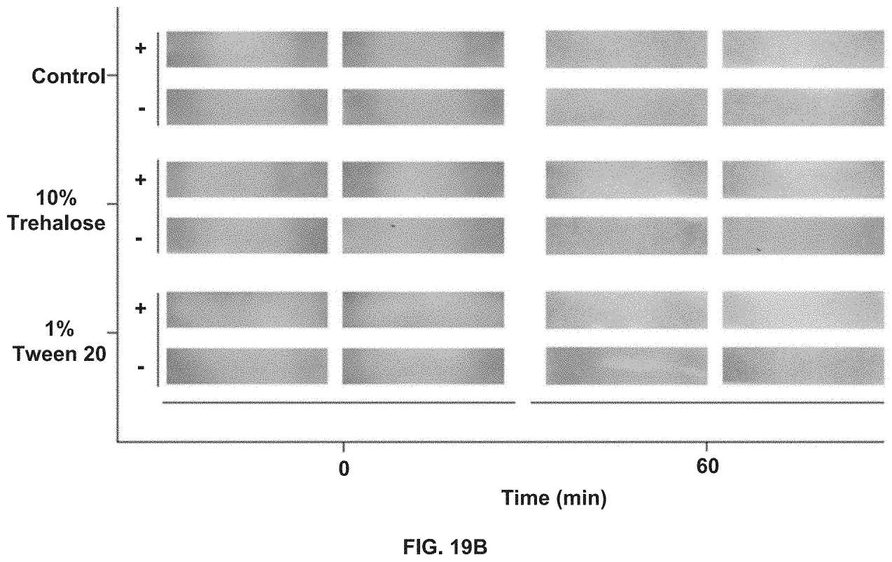

FIG. 19B shows colorimetric RT-LAMP results with the inclusion of Trehalose or Tween 20 at the given concentration. The orf1ab.II primer set was used. 20 μL of RT-LAMP master mix containing a base formulation of KCl (50 mM), MgSO₄ (8 mM), equimolar dNTP mixture (1.4 mM each dNTP), WarmStart BST 2.0 (0.32 U/μL), WarmStart RTx (0.3 U/μL), Phenol red (0.25 mM), dUTP (0.14 mM), Antarctic UDG (0.0004 U/μL), Tween 20 (1% v/v, if indicated), Betaine (20 mM), BSA (40 mg/mL), and Trehalose (10% w/v, if indicate) was added to Grade 1 chromatography paper (5 mm×20 mm) and allowed to dry inside a PCR preparation hood for 60 minutes. 25 μL of Heat-inactivated SARS-CoV-2 at a final concentration of 1×10⁵ copies per reaction in 25% processed saliva (positive reactions) or nuclease-free water (negative reactions) was added to the dry reaction pads. The pads were heated in an incubator set at 65° C. for 60 minutes and then scanned using a flatbed scanner.

Inclusion of ammonium sulfate caused a color from red to yellow upon drying of RT-LAMP reagents when no template was present. This color change was prevented by increasing the phenol red concentration and replacing ammonium sulfate with betaine (FIG. 19A). Furthermore, the addition of trehalose and bovine serum albumin (BSA) increased the reaction speed and increased LoD (FIG. 19B).

### EXAMPLE EMBODIMENTS

In one example there is provided, a composition for loop-mediated isothermal amplification (LAMP) analysis on a solid phase medium that includes or comprises one or more target primers; a DNA polymerase; and a re-solubilization agent; wherein the composition is substantially free of non-pH sensitive agents capable of discoloring the solid phase medium.

In one example of a composition for loop-mediated isothermal amplification (LAMP) analysis on a solid phase medium, the composition can further comprise an antioxidant.

In another example of a composition for loop-mediated isothermal amplification (LAMP) analysis on a solid phase medium, the composition can be substantially free of volatile agents.

In another example of a composition for loop-mediated isothermal amplification (LAMP) analysis on a solid phase medium, the composition can be substantially free of hygroscopic agents.

In another example of a composition for loop-mediated isothermal amplification (LAMP) analysis on a solid phase medium, hygroscopic agents can absorb more than about 10 wt % between about 40% and about 90% relative humidity (RH) at 25° C.

In another example of a composition for loop-mediated isothermal amplification (LAMP) analysis on a solid phase medium, the hygroscopic agents can include glycerol, ethanol, methanol, calcium chloride, potassium chloride, calcium sulfate, and combinations thereof.

In another example of a composition for loop-mediated isothermal amplification (LAMP) analysis on a solid phase medium, the re-solubilization agent can be a surfactant.

In another example of a composition for loop-mediated isothermal amplification (LAMP) analysis on a solid phase medium, the re-solubilization agent can comprise bovine serum albumin (BSA), casein, polysorbate 20, and combinations thereof.

In another example of a composition for loop-mediated isothermal amplification (LAMP) analysis on a solid phase medium, the target primers can target a pathogen comprising a viral pathogen, a bacterial pathogen, a fungal pathogen, or a protozoan pathogen.

In another example of a composition for loop-mediated isothermal amplification (LAMP) analysis on a solid phase medium, the pathogen can be a viral pathogen.

In another example of a composition for loop-mediated isothermal amplification (LAMP) analysis on a solid phase medium, the viral pathogen can comprise a dsDNA virus, an ssDNA virus, a dsRNA virus, a positive-strand ssRNA virus, a negative-strand ssRNA virus, an ssRNA-RT virus, or a ds-DNA-RT virus.

In another aspect, the viral pathogen can comprise H1N1, H2N2, H3N2, H1N1pdm09, or SARS-CoV-2.

In another example of a composition for loop-mediated isothermal amplification (LAMP) analysis on a solid phase medium, the composition can further comprise reverse transcriptase.

In another example of a composition for loop-mediated isothermal amplification (LAMP) analysis on a solid phase medium, the composition can further comprise a non-discoloration additive.

In another example of a composition for loop-mediated isothermal amplification (LAMP) analysis on a solid phase medium, the non-discoloration additive comprises one or more of a sugar, a buffer, or combinations thereof.

In another example of a composition for loop-mediated isothermal amplification (LAMP) analysis on a solid phase medium, the composition can further comprise an indicator.

In one example there is provided, a method for LAMP analysis on a solid phase medium comprising providing an assembly of a solid phase medium and a composition as recited herein, depositing a biological sample onto the solid phase medium, and heating the assembly to an isothermal temperature sufficient to facilitate a LAMP reaction.

In one example of a method for LAMP analysis on a solid phase medium, the biological sample is one or more of saliva, mucus, blood, urine, feces, sweat, exhaled breath condensate, and combinations thereof.

In another example of a method for LAMP analysis on a solid phase medium, the biological sample can be saliva.

In another example of a method for LAMP analysis on a solid phase medium, the method can further comprise detecting a viral pathogen.

In another example of a method for LAMP analysis on a solid phase medium, the LAMP analysis can be reverse transcriptase LAMP (RT-LAMP).

In one example there is provided, a system for performing the LAMP analysis including or comprising a composition as recited herein; and a solid phase medium on to which the composition is deposited.

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. A composition for loop-mediated isothermal amplification (LAMP) analysis on a solid phase medium comprising:
one or more target primers;
a DNA polymerase; and
a re-solubilization agent;
wherein the composition is substantially free of non-pH sensitive agents capable of discoloring the solid phase medium.

2. The composition of claim 1, further comprising an antioxidant.

3. The composition of claim 1, wherein the composition is substantially free of volatile agents.

4. The composition of claim 1, wherein the composition is substantially free of hygroscopic agents.

5. The composition of claim 4, wherein hygroscopic agents absorb more than about 10 wt % between about 40% and about 90% relative humidity (RH) at 25° C.

6. The composition of claim 4, wherein the hygroscopic agents include glycerol, ethanol, methanol, calcium chloride, calcium sulfate, and combinations thereof.

7. The composition of claim 1, wherein the re-solubilization agent is a surfactant.

8. The composition of claim 1, wherein the re-solubilization agent comprises bovine serum albumin (BSA), casein, polysorbate 20, and combinations thereof.

9. The composition of claim 1, wherein the target primers target a pathogen comprising a viral pathogen, a bacterial pathogen, a fungal pathogen, or a protozoan pathogen.

10. The composition of claim 9, wherein the pathogen is a viral pathogen.

11. The composition of claim 10, wherein the viral pathogen comprises a dsDNA virus, an ssDNA virus, a dsRNA virus, a positive-strand ssRNA virus, a negative-strand ssRNA virus, an ssRNA-RT virus, or a ds-DNA-RT virus.

12. The composition of claim 10, wherein the viral pathogen comprises H1N1, H2N2, H3N2, H1N1pdm09, or SARS-CoV-2.

13. The composition of claim 1, further comprising reverse transcriptase.

14. The composition of claim 1, further comprising a non-discoloration additive.

15. The composition of claim 14, wherein the non-discoloration additive comprises one or more of a sugar, a buffer, or combinations thereof.

16. The composition of claim 1, further comprising an indicator.

17. A method for LAMP analysis on a solid phase medium comprising:
providing an assembly of a solid phase medium and a composition as recited in claim 1;
depositing a biological sample onto the solid phase medium; and
heating the assembly to an isothermal temperature sufficient to facilitate a LAMP reaction.

18. The method of claim 17, wherein the biological sample is one or more of saliva, mucus, blood, urine, feces, and combinations thereof.

19. The method of claim 17, wherein the biological sample is saliva.

20. The method of claim 17, further comprising:
detecting a viral pathogen.

21. The method of claim 17, wherein the LAMP analysis is reverse transcriptase LAMP (RT-LAMP).

22. A system for performing the LAMP analysis comprising:
a composition as recited in claim 1; and
a solid phase medium on to which the composition is deposited.
